SEQUENCE LISTING (44 sequences)

SEQ ID NO: 1 - protein, 18 aa, Rattus norvegicus
  PEPTIDE (1)..(18): rat NogoA_342-357
SYDSIKLEPENPPPYEEA

SEQ ID NO: 2 - protein, 221 aa, Mus musculus
  CHAIN (1)..(221): Variable part of Heavy Chain of 3A6 with leader sequence
MDFGLIFFIVGLLKGVQCEVKLLESGGGLVQPGGSLKLSCVVSGFDFRRNWMSWVRQAPGKGLEWIGEINPDSSKINYTPSLKDKFIISRDNAKNTLYLQVSTVRSEDTALYYCVRPVWMYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVA

SEQ ID NO: 3 - protein, 238 aa, Mus musculus
  CHAIN (1)..(238): Light Chain of 3A6 with leader sequence
MSPAQFLFLLVLWIRETSGDVLLTQTPLTLSITIGQPASISCKSSQSLLHSDGKTYLNWLLQRPGQSPKRLIYLVSKLDSGVPDRFTGSGSGTDFTLKISRVEAEDLGLYYCWQGTHFPQTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 4 - DNA, 3919 bp, Homo sapiens
  CDS (1)..(3579): Human NogoA
atggaagacctggaccagtctcctctggtctcgtcctcggacagcccaccccggccgcagcccgcgttcaagtaccagttcgtgagggagcccgaggacgaggaggaagaagaggaggaggaagaggaggacgaggacgaagacctggaggagctggaggtgctggagaggaagcccgccgccgggctgtccgcggccccagtgcccaccgcccctgccgccggcgcgcccctgatggacttcggaaatgacttcgtgccgccggcgccccggggacccctgccggccgctccccccgtcgccccggagcggcagccgtcttgggacccgagcccggtgtcgtcgaccgtgcccgcgccatccccgctgtctgctgccgcagtctcgccctccaagctccctgaggacgacgagcctccggcccggcctccccctcctcccccggccagcgtgagcccccaggcagagcccgtgtggaccccgccagccccggctcccgccgcgcccccctccaccccggccgcgcccaagcgcaggggctcctcgggctcagtggatgagaccctttttgctcttcctgctgcatctgagcctgtgatacgctcctctgcagaaaatatggacttgaaggagcagccaggtaacactatttcggctggtcaagaggatttcccatctgtcctgcttgaaactgctgcttctcttccttctctgtctcctctctcagccgcttctttcaaagaacatgaataccttggtaatttgtcaacagtattacccactgaaggaacacttcaagaaaatgtcagtgaagcttctaaagaggtctcagagaaggcaaaaactctactcatagatagagatttaacagagttttcagaattagaatactcagaaatgggatcatcgttcagtgtctctccaaaagcagaatctgccgtaatagtagcaaatcctagggaagaaataatcgtgaaaaataaagatgaagaagagaagttagttagtaataacatccttcataatcaacaagagttacctacagctcttactaaattggttaaagaggatgaagttgtgtcttcagaaaaagcaaaagacagttttaatgaaaagagagttgcagtggaagctcctatgagggaggaatatgcagacttcaaaccatttgagcgagtatgggaagtgaaagatagtaaggaagatagtgatatgttggctgctggaggtaaaatcgagagcaacttggaaagtaaagtggataaaaaatgttttgcagatagccttgagcaaactaatcacgaaaaagatagtgagagtagtaatgatgatacttctttccccagtacgccagaaggtataaaggatcgttcaggagcatatatcacatgtgctccctttaacccagcagcaactgagagcattgcaacaaacatttttcctttgttaggagatcctacttcagaaaataagaccgatgaaaaaaaaatagaagaaaagaaggcccaaatagtaacagagaagaatactagcaccaaaacatcaaacccttttcttgtagcagcacaggattctgagacagattatgtcacaacagataatttaacaaaggtgactgaggaagtcgtggcaaacatgcctgaaggcctgactccagatttagtacaggaagcatgtgaaagtgaattgaatgaagttactggtacaaagattgcttatgaaacaaaaatggacttggttcaaacatcagaagttatgcaagagtcactctatcctgcagcacagctttgcccatcatttgaagagtcagaagctactccttcaccagttttgcctgacattgttatggaagcaccattgaattctgcagttcctagtgctggtgcttccgtgatacagcccagctcatcaccattagaagcttcttcagttaattatgaaagcataaaacatgagcctgaaaaccccccaccatatgaagaggccatgagtgtatcactaaaaaaagtatcaggaataaaggaagaaattaaagagcctgaaaatattaatgcagctcttcaagaaacagaagctccttatatatctattgcatgtgatttaattaaagaaacaaagctttctgctgaaccagctccggatttctctgattattcagaaatggcaaaagttgaacagccagtgcctgatcattctgagctagttgaagattcctcacctgattctgaaccagttgacttatttagtgatgattcaatacctgacgttccacaaaaacaagatgaaactgtgatgcttgtgaaagaaagtctcactgagacttcatttgagtcaatgatagaatatgaaaataaggaaaaactcagtgctttgccacctgagggaggaaagccatatttggaatcttttaagctcagtttagataacacaaaagataccctgttacctgatgaagtttcaacattgagcaaaaaggagaaaattcctttgcagatggaggagctcagtactgcagtttattcaaatgatgacttatttatttctaaggaagcacagataagagaaactgaaacgttttcagattcatctccaattgaaattatagatgagttccctacattgatcagttctaaaactgattcattttctaaattagccagggaatatactgacctagaagtatcccacaaaagtgaaattgctaatgccccggatggagctgggtcattgccttgcacagaattgccccatgacctttctttgaagaacatacaacccaaagttgaagagaaaatcagtttctcagatgacttttctaaaaatgggtctgctacatcaaaggtgctcttattgcctccagatgtttctgctttggccactcaagcagagatagagagcatagttaaacccaaagttcttgtgaaagaagctgagaaaaaacttccttccgatacagaaaaagaggacagatcaccatctgctatattttcagcagagctgagtaaaacttcagttgttgacctcctgtactggagagacattaagaagactggagtggtgtttggtgccagcctattcctgctgctttcattgacagtattcagcattgtgagcgtaacagcctacattgccttggccctgctctctgtgaccatcagctttaggatatacaagggtgtgatccaagctatccagaaatcagatgaaggccacccattcagggcatatctggaatctgaagttgctatatctgaggagttggttcagaagtacagtaattctgctcttggtcatgtgaactgcacgataaaggaactcaggcgcctcttcttagttgatgatttagttgattctctgaagtttgcagtgttgatgtgggtatttacctatgttggtgccttgtttaatggtctgacactactgattttggctctcatttcactcttcagtgttcctgttatttatgaacggcatcaggcacagatagatcattatctaggacttgcaaataagaatgttaaagatgctatggctaaaatccaagcaaaaatccctggattgaagcgcaaagctgaatgaaaacgcccaaaataattagtaggagttcatctttaaaggggatattcatttgattatacgggggagggtcagggaagaacgaaccttgacgttgcagtgcagtttcacagatcgttgttagatctttatttttagccatgcactgttgtgaggaaaaattacctgtcttgactgccatgtgttcatcatcttaagtattgtaagctgctatgtatggatttaaaccgtaatcatatctttttcctatctgaggcactggtggaataaaaaacctgtatattttactttgttgcagatagtcttgccgcatcttggcaagttgcagagatggtggagctag

SEQ ID NO: 5 - protein, 1192 aa, Homo sapiens
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 6 - protein, 18 aa, Homo sapiens
  PEPTIDE (1)..(18): Human NogoA_342-357
NYESIKHEPENPPPYEEA

SEQ ID NO: 7 - protein, 819 aa, Homo sapiens
  PEPTIDE (1)..(819): human Nig
DETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTS

SEQ ID NO: 8 - protein, 10 aa, Mus musculus
  BINDING (1)..(10): hypervariable part of heavy chain of 3A6
GFDFRRNWMS

SEQ ID NO: 9 - protein, 17 aa, Mus musculus
  BINDING (1)..(17): hypervariable part of heavy chain of 3A6
EINPDSSKINYTPSLKD

SEQ ID NO: 10 - protein, 9 aa, Mus musculus
  BINDING (1)..(9): hypervariable part of heavy chain of 3A6
PVWMYAMDY

SEQ ID NO: 11 - protein, 16 aa, Mus musculus
  BINDING (1)..(16): hypervariable part of light chain of 3A6
KSSQSLLHSDGKTYLN

SEQ ID NO: 12 - protein, 7 aa, Mus musculus
  BINDING (1)..(7): hypervariable part of light chain of 3A6
LVSKLDS

SEQ ID NO: 13 - protein, 9 aa, Mus musculus
  BINDING (1)..(9): hypervariable part of light chain of 3A6
WQGTHFPQT

SEQ ID NO: 14 - DNA, 30 bp, Mus musculus
  misc_binding (1)..(30): DNA-CDR-H1-3A6
ggattcgattttagaagaaattggatgagt

SEQ ID NO: 15 - DNA, 51 bp, Mus musculus
  misc_binding (1)..(51): DNA-CDR-H2-3A6
gaaattaatccagatagcagtaagataaactatacgccatctctaaaggat

SEQ ID NO: 16 - DNA, 27 bp, Mus musculus
  misc_binding (1)..(27): DNA-CDR-H3-3A6
ccggtctggatgtatgctatggactac

SEQ ID NO: 17 - DNA, 48 bp, Mus musculus
  misc_binding (1)..(48): DNA-CDR′1-3A6
aagtcaagtcagagcctcttgcatagtgatggaaagacatatttgaat

SEQ ID NO: 18 - DNA, 21 bp, Mus musculus
  misc_binding (1)..(21): DNA-CDR′2-3A6
ctggtgtctaaactggactct

SEQ ID NO: 19 - DNA, 27 bp, Mus musculus
  misc_binding (1)..(27): DNA-CDR′3-3A6
tggcaaggtacacattttcctcagacg

SEQ ID NO: 20 - DNA, 54 bp, Mus musculus
  CDS (1)..(54): leader sequence for heavy chain of 3A6
atggattttgggctgattttttttattgttggtcttttaaaaggggtccagtgt

SEQ ID NO: 21 - protein, 18 aa, Mus musculus
MDFGLIFFIVGLLKGVQC

SEQ ID NO: 22 - DNA, 57 bp, Mus musculus
  CDS (1)..(57): leader sequence for 3A6-light chain
atgagtcctgcccagttcctgtttctgttagtgctctggattcgggaaaccagcggt

SEQ ID NO: 23 - protein, 19 aa, Mus musculus
MSPAQFLFLLVLWIRETSG

SEQ ID NO: 24 - protein, 181 aa, Homo sapiens
  PEPTIDE (1)..(181): human Nig-D20
GTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLF

SEQ ID NO: 25 - DNA, 3492 bp, Rattus norvegicus
  CDS (1)..(3492): rat NogoA
atggaagacatagaccagtcgtcgctggtctcctcgtccacggacagcccgccccggcctccgcccgccttcaagtaccagttcgtgacggagcccgaggacgaggaggacgaggaggaggaggaggacgaggaggaggacgacgaggacctagaggaactggaggtgctggagaggaagcccgcagccgggctgtccgcagctgcggtgccgcccgccgccgccgcgccgctgctggacttcagcagcgactcggtgccccccgcgccccgcgggccgctgccggccgcgccccctgccgctcctgagaggcagccatcctgggaacgcagccccgcggcgcccgcgccatccctgccgcccgctgccgcagtcctgccctccaagctcccagaggacgacgagcctccggcgaggcccccgcctccgccgccagccggcgcgagccccctggcggagcccgccgcgcccccttccacgccggccgcgcccaagcgcaggggctccggctcagtggatgagaccctttttgctcttcctgctgcatctgagcctgtgataccctcctctgcagaaaaaattatggatttgatggagcagccaggtaacactgtttcgtctggtcaagaggatttcccatctgtcctgcttgaaactgctgcctctcttccttctctatctcctctctcaactgtttcttttaaagaacatggataccttggtaacttatcagcagtgtcatcctcagaaggaacaattgaagaaactttaaatgaagcttctaaagagttgccagagagggcaacaaatccatttgtaaatagagatttagcagaattttcagaattagaatattcagaaatgggatcatcttttaaaggctccccaaaaggagagtcagccatattagtagaaaacactaaggaagaagtaattgtgaggagtaaagacaaagaggatttagtttgtagtgcagcccttcacagtccacaagaatcacctgtgggtaaagaagacagagttgtgtctccagaaaagacaatggacatttttaatgaaatgcagatgtcagtagtagcacctgtgagggaagagtatgcagactttaagccatttgaacaagcatgggaagtgaaagatacttatgagggaagtagggatgtgctggctgctagagctaatgtggaaagtaaagtggacagaaaatgcttggaagatagcctggagcaaaaaagtcttgggaaggatagtgaaggcagaaatgaggatgcttctttccccagtaccccagaacctgtgaaggacagctccagagcatatattacctgtgcttcctttacctcagcaaccgaaagcaccacagcaaacactttccctttgttagaagatcatacttcagaaaataaaacagatgaaaaaaaaatagaagaaaggaaggcccaaattataacagagaagactagccccaaaacgtcaaatcctttccttgtagcagtacaggattctgaggcagattatgttacaacagataccttatcaaaggtgactgaggcagcagtgtcaaacatgcctgaaggtctgacgccagatttagttcaggaagcatgtgaaagtgaactgaatgaagccacaggtacaaagattgcttatgaaacaaaagtggacttggtccaaacatcagaagctatacaagaatcactttaccccacagcacagctttgcccatcatttgaggaagctgaagcaactccgtcaccagttttgcctgatattgttatggaagcaccattaaattctctccttccaagcgctggtgcttctgtagtgcagcccagtgtatccccactggaagcacctcctccagttagttatgacagtataaagcttgagcctgaaaaccccccaccatatgaagaagccatgaatgtagcactaaaagctttgggaacaaaggaaggaataaaagagcctgaaagttttaatgcagctgttcaggaaacagaagctccttatatatccattgcgtgtgatttaattaaagaaacaaagctctccactgagccaagtccagatttctctaattattcagaaatagcaaaattcgagaagtcggtgcccgaacacgctgagctagtggaggattcctcacctgaatctgaaccagttgacttatttagtgatgattcgattcctgaagtcccacaaacacaagaggaggctgtgatgctcatgaaggagagtctcactgaagtgtctgagacagtagcccagcacaaagaggagagacttagtgcctcacctcaggagctaggaaagccatatttagagtcttttcagcccaatttacatagtacaaaagatgctgcatctaatgacattccaacattgaccaaaaaggagaaaatttctttgcaaatggaagagtttaatactgcaatttattcaaatgatgacttactttcttctaaggaagacaaaataaaagaaagtgaaacattttcagattcatctccgattgagataatagatgaatttcccacgtttgtcagtgctaaagatgattctcctaaattagccaaggagtacactgatctagaagtatccgacaaaagtgaaattgctaatatccaaagcggggcagattcattgccttgcttagaattgccctgtgacctttctttcaagaatatatatcctaaagatgaagtacatgtttcagatgaattctccgaaaataggtccagtgtatctaaggcatccatatcgccttcaaatgtctctgctttggaacctcagacagaaatgggcagcatagttaaatccaaatcacttacgaaagaagcagagaaaaaacttccttctgacacagagaaagaggacagatccctgtcagctgtattgtcagcagagctgagtaaaacttcagttgttgacctcctctactggagagacattaagaagactggagtggtgtttggtgccagcttattcctgctgctgtctctgacagtgttcagcattgtcagtgtaacggcctacattgccttggccctgctctcggtgactatcagctttaggatatataagggcgtgatccaggctatccagaaatcagatgaaggccacccattcagggcatatttagaatctgaagttgctatatcagaggaattggttcagaaatacagtaattctgctcttggtcatgtgaacagcacaataaaagaactgaggcggcttttcttagttgatgatttagttgattccctgaagtttgcagtgttgatgtgggtgtttacttatgttggtgccttgttcaatggtctgacactactgattttagctctgatctcactcttcagtattcctgttatttatgaacggcatcaggtgcagatagatcattatctaggacttgcaaacaagagtgttaaggatgccatggccaaaatccaagcaaaaatccctggattgaagcgcaaagcagattga

SEQ ID NO: 26 - protein, 1163 aa, Rattus norvegicus
MEDIDQSSLVSSSTDSPPRPPPAFKYQFVTEPEDEEDEEEEEDEEEDDEDLEELEVLERKPAAGLSAAAVPPAAAAPLLDFSSDSVPPAPRGPLPAAPPAAPERQPSWERSPAAPAPSLPPAAAVLPSKLPEDDEPPARPPPPPPAGASPLAEPAAPPSTPAAPKRRGSGSVDETLFALPAASEPVIPSSAEKIMDLMEQPGNTVSSGQEDFPSVLLETAASLPSLSPLSTVSFKEHGYLGNLSAVSSSEGTIEETLNEASKELPERATNPFVNRDLAEFSELEYSEMGSSFKGSPKGESAILVENTKEEVIVRSKDKEDLVCSAALHSPQESPVGKEDRVVSPEKTMDIFNEMQMSVVAPVREEYADFKPFEQAWEVKDTYEGSRDVLAARANVESKVDRKCLEDSLEQKSLGKDSEGRNEDASFPSTPEPVKDSSRAYITCASFTSATESTTANTFPLLEDHTSENKTDEKKIEERKAQIITEKTSPKTSNPFLVAVQDSEADYVTTDTLSKVTEAAVSNMPEGLTPDLVQEACESELNEATGTKIAYETKVDLVQTSEAIQESLYPTAQLCPSFEEAEATPSPVLPDIVMEAPLNSLLPSAGASVVQPSVSPLEAPPPVSYDSIKLEPENPPPYEEAMNVALKALGTKEGIKEPESFNAAVQETEAPYISIACDLIKETKLSTEPSPDFSNYSEIAKFEKSVPEHAELVEDSSPESEPVDLFSDDSIPEVPQTQEEAVMLMKESLTEVSETVAQHKEERLSASPQELGKPYLESFQPNLHSTKDAASNDIPTLTKKEKISLQMEEFNTAIYSNDDLLSSKEDKIKESETFSDSSPIEIIDEFPTFVSAKDDSPKLAKEYTDLEVSDKSEIANIQSGADSLPCLELPCDLSFKNIYPKDEVHVSDEFSENRSSVSKASISPSNVSALEPQTEMGSIVKSKSLTKEAEKKLPSDTEKEDRSLSAVLSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNSTIKELRRLFLVDDLVDSLKFAVLMWVFTYVGALFNGLTLLILALISLFSIPVIYERHQVQIDHYLGLANKSVKDAMAKIQAKIPGLKRKAD

SEQ ID NO: 27 - protein, 25 aa, Rattus norvegicus
  PEPTIDE (1)..(25): rat PEP4
EELVQKYSNSALGHVNSTIKELRRL

SEQ ID NO: 28 - protein, 17 aa, Artificial Sequence
  PRO/SER rich peptide
PSSPPPSSPPPSSPPPS

SEQ ID NO: 29 - DNA, 25 bp, Artificial Sequence
  CA-NA-2F
aagcaccattgaattctgcagttcc

SEQ ID NO: 30 - DNA, 28 bp, Artificial Sequence
  CA-NA-3R primer
aactgcagtactgagctcctccatctgc

SEQ ID NO: 31 - DNA, 33 bp, Artificial Sequence
  forward 5′
gtcgcggatccatggagaccctttttgctcttc

SEQ ID NO: 32 - DNA, 27 bp, Artificial Sequence
  reverse 5′
gttctcgagttatgaagttttactcag

SEQ ID NO: 33 - DNA, 29 bp, Artificial Sequence
  forward 5′-1
gtgcggatccatggatttgaaggagcagc

SEQ ID NO: 34 - DNA, 28 bp, Artificial Sequence
  reverse 5′-1
gtttctcgagtgaagttttattcagctc

SEQ ID NO: 35 - DNA, 20 bp, Artificial Sequence
  5′ primer
tccaccccggccgcgcccaa

SEQ ID NO: 36 - DNA, 22 bp, Artificial Sequence
  5′ primer 2
aatgatgggcaaagctgtgctg

SEQ ID NO: 37 - DNA, 24 bp, Artificial Sequence
  3′ primer
ggtacaaagattgcttatgaaaca

SEQ ID NO: 38 - DNA, 22 bp, Artificial Sequence
  3′ primer 2
agcagggccaaggcaatgtagg

SEQ ID NO: 39 - DNA, 28 bp, Artificial Sequence
  5′-VL leader
aatatgagtcctgcccagttcctgtttc

SEQ ID NO: 40 - DNA, 32 bp, Artificial Sequence
  3′-Ck
ttaggaattcctaacactctcccctgttgaag

SEQ ID NO: 41 - DNA, 31 bp, Artificial Sequence
  5′-VH leader
aatatggattttgggctgattttttttattg

SEQ ID NO: 42 - DNA, 24 bp, Artificial Sequence
  3′-CH hinge
aattgggcaacgttgcaggtgacg

SEQ ID NO: 43 - DNA, 663 bp, Mus musculus
  misc_binding (1)..(663): DNA variable part of heavy chain 3A6
atggattttgggctgattttttttattgttggtcttttaaaaggggtccagtgtgaggtgaagcttctcgagtctggaggtggcctggtgcagcctggaggatccctgaaactctcctgtgtagtctcaggattcgattttagaagaaattggatgagttgggtccggcaggctcctgggaaagggctagaatggattggagaaattaatccagatagcagtaagataaactatacgccatctctaaaggataaattcatcatctccagagacaatgccaagaatacgctgtacctgcaagtgagcacagtgagatctgaggacacagccctttattactgtgtgagaccggtctggatgtatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcc

SEQ ID NO: 44 - DNA, 717 bp, Mus musculus
  misc_binding (1)..(717): variable part of light chain of 3A6
atgagtcctgcccagttcctgtttctgttagtgctctggattcgggaaaccagcggtgatgttctgttgacccagactcctctcactttgtcgataaccattggacaaccagcctccatctcttgcaagtcaagtcagagcctcttgcatagtgatggaaagacatatttgaattggttgttacagaggccaggccagtctccaaagcgcctaatctatctggtgtctaaactggactctggagtccctgacaggttcactggcagtggatcagggacggatttcacactgaaaatcagcagagtggaggctgaggatttgggactttattattgctggcaaggtacacattttcctcagacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttag

(19) United States
(12) Patent Application Publication
(10) Pub. No.: US 2011/0008334 A1
(43) Pub. Date: Jan. 13, 2011

(54) NOGO-A BINDING WITH ENHANCED AFFINITY AND PHARMACEUTICAL USE THEREOF

(75) Inventors: Carmen Barske, Loerrach (DE); Stefan Frentzel, Lörrach (DE); Anis Khusro Mir, Bartenheim (FR); Martin E. Schwab, Zurich (CH); Alessandra Vitaliti, Bedigliora (CH)

Correspondence Address: NOVARTIS;CORPORATE INTELLECTUAL PROPERTY, ONE HEALTH PLAZA 101/2, EAST HANOVER, NJ, 07936-1080, US

(73) Assignee: Novartis Pharma AG, Basel (CH)

(21) Appl. No.: 12/816,964

(22) Filed: Jun. 16, 2010

Related U.S. Application Data
(63) Continuation of application No. 10/572,434, filed on Jun. 2, 2006, now abandoned.

(30) Foreign Application Priority Data
Sep. 17, 2003 (GB) 0321997.9
Sep. 17, 2004 (EP) PCT/EP2004/010489

(51) Int. Cl. A61K 39/395; C07K 16/18; C07K 7/06; C07K 7/08; A61P 25/00; A61P 25/02

(52) U.S. Cl. 424/133.1; 530/387.3; 530/387.9; 424/139.1; 530/328; 530/326

(57) ABSTRACT

The present invention provides a binding molecule which is capable of binding to the human NogoA polypeptide or human NiG or human NiG-D20 or human NogoA_342-357 with a dissociation constant, 1000 nM, a polynucleotide encoding such binding molecule; an expression vector comprising said polynucleotide capable of producing a binding molecule; an isolated host cell which comprises an expression system as defined above; the use of such binding molecule as a pharmaceutical, especially in the treatment of a nerve repair, a pharmaceutical composition comprising said binding molecule; and a method of treatment of nerve repair; a pharmaceutical composition comprising said binding molecule; and a method of treatment of diseases associated with nerve repair.

### RELATED APPLICATIONS

This present invention is a continuation patent application that claims priority to U.S. patent application Ser. No. 10/572,434, filed on Mar. 17, 2006, PCT, which claims priority to PCT patent application number PCT/EP2004/010489, filed Sep. 17, 2004, which claims priority to Great Britain application No. 0321997.9 filed on Sep. 19, 2003, the entirety of which are herein incorporated by reference.

This invention relates to NogoA binding molecules, such as for example monoclonal antibodies or Fab fragments thereof.

Neuronal regeneration following injury in the adult central nervous system (CNS) is limited due to the presence of the inhibitory myelin environment that ensheaths axons and formation of scar tissue. In the last few years important insights have been gained into the molecular understanding why the CNS is unable to spontaneously repair itself following injury. Inhibitory molecules in the myelin are the major impediment for the axonal regeneration, particularly immediately after the injury. So far NogoA, Myelin-Associated Glycoprotein (MAG) and myelin-oligodendrocyte glycoprotein (OMgp) have been characterised as potent inhibitors of neurite outgrowth. In addition, myelin also contains other inhibitory components, such as, chondroitin sulphate proteoglycans. Nogo-A is a member of the reticulon protein family and it has at least two biologically active and pharmacologically distinct domains termed Amino-Nogo and Nogo-66. While the receptor site for the former is not known so far, Nogo-66 inhibits neuronal growth in vitro and in vivo via the neuronal receptor NgR. In addition to Nogo-66, MAG and OMgp also bind to the NgR with high affinity and inhibit neurite outgrowth.

Potential new research approaches currently pursued for enhancement of nerve repair include digestion of scar tissue using an enzyme chondroitinase ABC, bridging techniques using Olfactory ensheathing cells and stem cells and protein growth factors to boost neuronal growth. Blocking actions of neurite outgrowth inhibitors by modulation of intracellular signalling mediators such as Rho, a membrane-bound guanosine tris-phosphatase (GTPase), which appears to be a key link in the inhibition of axonal growth. Cyclic adenosine monophosphate (cAMP) which can overcome myelin associated inhibition in vitro and induce regeneration in vivo. Use of peptide inhibitor of the NgR receptor (NEP 1-40) to induce neuronal regrowth and functional recovery in rats following spinal injury.

In addition to the use of the approaches described above, attention has also focused upon the use of certain monoclonal antibodies to neutralize neurite growth inhibitory molecules of the central and peripheral nervous system, in particular to neutralize the neurite growth inhibitory activity of NogoA. Thus it has been shown that the monoclonal antibody IN-1 or the IN-1 Fab fragment thereof induce neurite outgrowth in vitro and enhance sprouting and regeneration in vivo (Schwab M E et al. (1996) Physiol. Rev. 76, 319-370). Testing different domains of the NogoA for neurite growth inhibitory activity have delineated several inhibitory domains in the molecule (Chen et al. (2000) Nature 403, 434-439; GrandPre et al. (2000) Nature 403, 439-444; Prinjha et al. (2000) Nature 403, 383-384. Natural immunoglobulins or antibodies comprise a generally Y-shaped multimeric molecule having an antigen-binding site at the end of each upper arm. The remainder of the structure, in particular the stem of the Y mediates effector functions associated with the immunoglobulins. Antibodies consists of a 2 heavy and 2 light chains. Both heavy and light chains comprise a variable domain and a constant part. An antigen binding site consists of the variable domain of a heavy chain associated with the variable domain of a light chain. The variable domains of the heavy and light chains have the same general structure. More particularly, the antigen binding characteristics of an antibody are essentially determined by 3 specific regions in the variable domain of the heavy and light chains which are called hypervariable regions or complementarity determining regions (CDRs). These 3 hypervariable regions alternate with 4 framework regions (FRs) whose sequences are relatively conserved and which are not directly involved in binding. The CDRs form loops and are field in close proximity by the framework regions which largely adopt a β-sheet conformation. The CDRs of a heavy chain together with the CDRs of the associated light chain essentially constitute the antigen binding site of the antibody molecule. The determination as to what constitutes an FR or a CDR region is usually made by comparing the amino acid sequence of a number of antibodies raised in the same species. The general rules for identifying the CDR and FR regions are general knowledge of a man skilled in the art and can for example be found in the website (http://www.bioinf.org.uk/abs/).

It has now surprisingly been found that a novel monoclonal human antibody (hereinafter called “3A6”) raised in Medarex Mice (genetically reconstituted mice with human immunoglobulin genes) against human NiG and of the IgG type has better properties than the NogoA antibodies of the prior art (Schwab M E et al. (1996) Physiol. Rev. 76, 319-370), especially with regard to the binding affinity to NogoA of different species including the homo sapiens and with regard to its higher Nogo-A neurite outgrowth neutralizing activity at a given antibody concentration. Moreover it is now possible to construct other NogoA binding molecules having the same hypervariable regions as said antibody.

Accordingly, the invention provides binding molecules to a particular region or epitope of NogoA (hereinafter referred to as “the Binding Molecules of the invention” or simply “Binding Molecules”). Preferably, the Binding Molecules of the invention bind human NogoA_{—}342-357 (epitope of 3A6 in human NiG; =SEQ ID NO: 6), human NogoA (SEQ ID NO: 5; or human NiG (which is the most potent neurite outgrowth inhibitory fragment of NogoA and starts at amino acid No. 186 and ends at amino acid No. 1004 of human NogoA, = SEQ ID NO: 5) with a dissociation constant (Kd)<1000 nM, more preferably with a Kd<100 nM, most preferably with a Kd<10 nM. The binding reaction may be shown by standard methods (qualitative assays) including, for example, the ELISA method described in Example 6 and the biosensor affinity method described in the Example 7. In addition, the binding to human NogoA and almost more importantly the efficiency may be shown in a neurite outgrowth assay, e.g. as described below.

Thus, in a further preferred embodiment the Binding Molecules (at a concentration of 100 μg/ml, preferably 100 μg/ml, more preferably at 1.0 μg/ml even more preferably at 0.1 μg/ml) enhance the number of neurites of rat cerebellar granule cells on a substrate of monkey brain protein extract by at least 20%, preferably 50%, most preferred 80% compared to the number of neurites of rat cerebellar granule cells which are treated with a control antibody that does not bind to the human NogoA, human NiG or NogoA_{—}342-357 polypeptide (i.e. that has a dissociation constant>1000 nM).

In a further preferred embodiment the Binding Molecules of the invention comprises at least one antigen binding site, said antigen binding site comprising in sequence, the hypervariable regions CDR-H1-3A6, CDR-H2-3A6 and CDR-H3-3A6; said CDR-H1-3A6 having the amino acid sequence SEQ ID NO: 8, said CDR-H2-3A6 having the amino acid sequence SEQ ID NO: 9, and said CDR-H3-3A6 having the amino acid sequence SEQ ID NO: 10; and direct equivalents thereof.

In a further aspect of the invention, the Binding Molecule of the invention comprises at least one antigen binding site, said antigen binding site comprising either
a) in sequence the hypervariable regions CDR-H1-3A6, CDR-H2-3A6 and CDR-H3-3A6; said CDR-H1-3A6 having the amino acid sequence of SEQ ID NO: 8, said CDR-H2-3A6 having the amino acid sequence of SEQ ID NO: 9, and said CDR-H3-3A6 having the amino acid sequence SEQ ID NO: 10; or
b) in sequence the hypervariable regions CDR-L1-3A6, CDR-L2-3A6 and CDR-L3-3A6, said CDR-L1-3A6 having the amino acid sequence of SEQ ID NO: 11, said CDR-L2-3A6 having the amino acid sequence of SEQ ID NO: 12, and said CDR-L3-3A6 having the amino acid sequence of SEQ ID NO: 13; or
c) direct equivalents thereof.

In a further aspect of the invention, the Binding Molecule of the invention comprises at least
a) a first domain comprising in sequence the hypervariable regions CDR-H1-3A6, CDR-H2-3A6 and CDR-H3-3A6; said CDR-H1-3A6 having the amino acid sequence of SEQ ID NO: 8, said CDR-H2-3A6 having the amino acid sequence of SEQ ID NO: 9, and said CDR-H3-3A6 having the amino acid sequence SEQ ID NO: 10; and
b) a second domain comprising in sequence the hypervariable regions CDR-L1-3A6, CDR-L2-3A6 and CDR-L3-3A6, said CDR-L1-3A6 having the amino acid sequence of SEQ ID NO: 11, said CDR-L2-3A6 having the amino, acid sequence of SEQ ID NO: 12, and said CDR-L3-3A6 having the amino acid sequence of SEQ ID NO: 13; or
c) direct equivalents thereof.

Moreover, the invention also provides the following Binding Molecule of the invention, which comprises at least one antigen binding site comprising
a) either the variable part of the heavy chain of 3A6 (SEQ ID NO: 2); or
b) the variable part of the light chain of 3A6 (SEQ ID NO: 3), or direct equivalents thereof.

When the antigen binding site comprises both the first and second domains, these may be located on the same polypeptide molecule or, preferably, each domain may be on a different chain, the first domain being part of an immunoglobulin heavy chain or fragment thereof and the second domain being part of an immunoglobulin light chain or fragment thereof.

Examples of Binding Molecules of the invention include antibodies as produced by B-cells or hybridomas and human or chimeric or humanized antibodies or any fragment thereof, e.g. F(ab′)₂; and Fab fragments, as well as single chain or single domain antibodies.

A single chain antibody consists of the variable domains of an antibody heavy and light chains covalently bound by a peptide linker usually consisting of from 10 to 30 amino acids, preferably from 15 to 25 amino acids. Therefore, such a structure does not include the constant part of the heavy and light chains and it is believed that the small peptide spacer should be less antigenic than a whole constant part. By “chimeric antibody” is meant an antibody in which the constant regions of heavy or light chains or both are of human origin while the variable domains of both heavy and light chains are of non-human (e.g. murine) origin. By “humanized antibody” is meant an antibody in which the hypervariable regions (CDRs) are of non-human (e.g. murine) origin, while all or substantially all the other parts of the immunoglobulin e.g. the constant regions and the highly conserved parts of the variable domains, i.e. the framework regions, are of human origin. A humanized antibody may however retain a few amino acids of the murine sequence in the parts of the framework regions adjacent to the hypervariable regions.

Hypervariable regions may be associated with any kind of framework regions, preferably of murine or human origin. Suitable framework regions are described in “Sequences of proteins of immunological interest”, Kabat E. A. et al, US department of health and human services, Public health service, National Institute of Health. Preferably the constant part of a human heavy chain of the Binding Molecules may be of the IgG4 type, including subtypes, preferably the constant part of a human light chain may be of the κ or λ type, more preferably of the κ type.

Monoclonal antibodies raised against a protein naturally found in all humans may be developed in a non-human system e. g. in mice. As a direct consequence of this, a xenogenic antibody as produced by a hybridoma, when administered to humans, elicits an undesirable immune response, which is predominantly mediated by the constant part of the xenogenic immunoglobulin. This clearly limits the use of such antibodies as they cannot be administered over a prolonged period of time. Therefore it is particularly preferred to use single chain, single domain, chimeric or humanized antibodies which are not likely to elicit a substantial allogenic response when administered to humans.

In view of the foregoing, a more preferred Binding Molecule of the invention is selected from a chimeric antibody, which comprises at least
a) one immunoglobulin heavy chain or fragment thereof which comprises (i) a variable domain comprising in sequence the hypervariable regions CDR-H1-3A6, CDR-H2-3A6 and CDR-H3-3A6 and (ii) the constant part or fragment thereof of a human heavy chain; said CDR-H1-3A6 having the amino acid sequence (SEQ ID NO: 8), said CDR-H2-3A6 having the amino acid sequence (SEQ ID NO: 9), and said CDR-H3-3A6 having the amino-acid sequence (SEQ ID NO: 10), and
b) one immunoglobulin light chain or fragment thereof which comprises (i) a variable domain comprising in sequence the hypervariable regions CDR-L1-3A6, CDR-L2-3A6 and CDR-L3-3A6 and (ii) the constant part or fragment thereof of a human light chain; said CDR-L1-3A6 having the amino acid sequence (SEQ ID NO: 11), said CDR-L2-3A6 having the amino acid sequence (SEQ ID NO: 12), and said CDR-L3-3A6 having the amino acid sequence (SEQ ID NO: 13); or
   direct equivalents thereof.

Alternatively, a Binding Molecule of the invention may be selected from a single chain binding molecule Which comprises an antigen binding site comprising
a) a first domain comprising in sequence the hypervariable CDR-H1-3A6, CDR-H2-3A6 and CDR-H3-3A6; said CDR-H1-3A6 having the amino acid sequence (SEQ ID NO: 8), said CDR-H2-3A6 having the amino acid sequence (SEQ ID NO: 9), and said CDR-H3-3A6 having the amino acid sequence (SEQ ID NO: 10); and
b) a second domain comprising in sequence the hypervariable CDR-L1-3A6, CDR-L2-3A6 and CDR-L3-3A6; said CDR-L1-3A6 having the amino acid sequence (SEQ ID NO: 11), said CDR-L2-3A6 having the amino acid sequence (SEQ ID NO: 12), and said CDR-L3-3A6 having the amino acid sequence (SEQ ID NO: 13); and
c) a peptide linker which is bound either to the N-terminal extremity of the first domain and to the C-terminal extremity of the second domain or to the C-terminal extremity of the first domain and to the N-terminal extremity of second domain;
   or direct equivalents thereof.

As it is well known, minor changes in an amino acid sequence such as deletion, addition or substitution of one or several amino acids may lead to an allelic form of the original protein which has substantially identical properties. Thus, by the term “direct equivalents thereof” is meant either any single domain Binding Molecule of the invention (molecule X)
(i) in which each of the hypervariable regions CDR-H1, CDR-H2, and CDR-H3 of the Binding Molecule is at least 50 or 80% homologous, preferably at least 90% homologous, more preferably at least 95, 96, 97, 98, 99% homologous to the equivalent hypervariable regions of CDR-H1-3A6 (SEQ ID NO: 8), CDR-H2-3A6 (SEQ ID NO: 9) and CDR-H3-3A6 (SEQ ID NO: 10), whereas CDR-H1 is equivalent to CDR-H1-3A6, CDR-H2 is equivalent to CDR-H2-3A6, CDR-H3 is equivalent to CDR-H3-3A6; and
(ii) which is capable of binding to the human NogoA, human NiG, or human NogoA_{—}342-357, preferably with a dissociation constant (Kd)<1000 nM, more preferably with a Kd<100 nM, most preferably with a Kd<10 nM, or
   any binding molecule of the invention having at least two domains per binding site (molecule X′)
(iii) in which each of the hypervariable regions CDR-H1, CDR-H2, CDR-H3, CDR-L1, CDR-L2 and CDR-L3 is at least 50 or 80% homologous, preferably at least 90% homologous, more preferably at least 95, 96,97, 98, 99% identical to the equivalent hypervariable regions of CDR-H1-3A6 (SEQ ID NO: 8), CDR-H2-3A6 (SEQ ID NO: 9), CDR-H3-3A6 (SEQ ID NO: 10), CDR-L1-3A6 (SEQ ID NO: 11), CDR-L2-3A6 (SEQ ID NO: 12), and CDR-L3-3A6 (SEQ ID NO: 13), whereas CDR-H1 is equivalent to CDR-H1-3A6, CDR-H2is equivalent to CDR-H2-3A6, CDR-H3 is equivalent to CDR-H3-3A6, CDR-L1 is equivalent to CDR-L1-3A6, CDR-L2 is equivalent to CDR-L2-3A6, CDR-L3 is equivalent to CDR-L3-3A6; and
(iv) which is capable of binding the human NogoA, human NiG, or human NogoA_{—}342-357, preferably with a dissociation constant (Kd)<1000 nM, more preferably with a Kd<100 nM, most preferably with a Kd<10 nM.

Thus further embodiments of the inventions are for example a Binding Molecule which is capable of binding to the human NogoA, human NiG, or human NogoA_{—}342-357 with a dissociation constant<1000 nM and comprises at least one antigen binding site, said antigen binding site comprising either
in sequence the hypervariable regions CDR-H1, CDR-H2, and CDR-H3, of which each of the hypervariable regions are at least 50%, preferably 80, 90, 95, 96, 97, 98, 99% homologous to their equivalent hypervariable regions CDR-H1-3A6 (SEQ ID NO: 8), CDR-H2-3A6 (SEQ ID NO: 9) and CDR-H3-3A6 (SEQ ID NO: 10); or
in sequence the hypervariable regions CDR-L1, CDR-L2, and CDR-L3, of which each of the hypervariable regions are at least 50%, preferably 80, 90, 95, 96, 97, 98, 99% homologous to their equivalent hypervariable regions CDR-L1-3A6 (SEQ ID NO: 11), CDR-L2-3A6 (SEQ ID NO: 12) and CDR-L3-3A6 (SEQ ID NO: 13).

Furthermore, a Binding Molecule which is capable of binding the human NogoA, human NiG, or human NogoA_{—}342-357 with a dissociation constant<1000 nM and comprises
a first antigen binding site comprising in sequence the hypervariable regions CDR-H1, CDR-H2, and CDR-H3, of which each of the hypervariable regions are at least 50%, preferably 80, 90, 95,96, 97, 98, 99% homologous to their equivalent hypervariable regions CDR-H1-3A6 (SEQ ID NO: 8), CDR-H2-3A6 (SEQ ID NO: 9) and CDR-H3-3A6 (SEQ ID NO: 10); and
a second antigen binding site comprising in sequence the hypervariable regions CDR-L1, CDR-L2, and CDR-L3, of which each of the hypervariable regions are at least 50%, preferably 80, 90, 95, 96, 97, 98, 99% homologous to their equivalent hypervariable regions CDR-L1-3A6 (SEQ ID NO: 11), CDR-L2-3A6 (SEQ ID NO: 12) and CDR-L3-3A6 (SEQ ID NO: 13).

This dissociation constant may be conveniently tested in various assays including, for example, the biosensor affinity method described in Example 7, in addition, the binding and functional effect of the Binding Molecules may be shown in a bioassay, e.g. as described below.

The constant part of a human heavy chain may be of the γ1; γ2; γ3; γ4; α1; α2; δ or ε type, preferably of the γ type, more preferably of the γ4; type, whereas the constant part of a human light chain may be of the κ or λ type (which includes the λ1; λ2; and λ3 subtypes) but is preferably of the κ type. The amino acid sequence of all these constant parts are given in Kabat et al (Supra).

Conjugates of the binding molecules of the invention, e. g. enzyme or toxin or radioisotope conjugates, are also included within the scope of the invention.

“Polypeptide”, if not otherwise specified herein, includes any peptide or protein comprising amino acids joined to each other by peptide bonds, having an amino acid sequence starting at the N-terminal extremity and ending at the C-terminal extremity. Preferably, the polypeptide of the present invention is a monoclonal antibody, more preferred is a chimeric (also called V-grafted) or humanised (also called CDR-grafted) monoclonal antibody. The humanised (CDR-grafted) monoclonal antibody may or may not include further mutations introduced into the framework (FR) sequences of the acceptor antibody.

A functional derivative of a polypeptide as used herein includes a molecule having a qualitative biological activity in common with a polypeptide to the present invention, i.e. having the ability to bind to the human NogoA, human NiG, or human NogoA_{—}342-357. A functional derivative includes fragments and peptide analogs of a polypeptide according to the present invention. Fragments comprise regions within the sequence of a polypeptide according to the present invention, e.g. of a specified sequence. The term “derivative” is used to define amino acid sequence variants, and covalent modifications of a polypeptide according to the present invention, e.g. of a specified sequence. The functional derivatives of a polypeptide according to the present invention, e.g. of a specified sequence, e.g. of the hypervariable region of the light and the heavy chain, preferably have at least about 65%, more preferably at least about 75%, even more preferably at least about 85%, most preferably at least about 95, 96, 97, 98, 99% overall sequence homology with the amino acid sequence of a polypeptide according to the present invention, e.g. of a specified sequence, and substantially retain the ability to bind the human NogoA, human NiG or human NogoA_{—b 342}-357.

The term “covalent modification” includes modifications of a polypeptide according to the present invention, e.g. of a specified sequence; or a fragment thereof with an organic proteinaceous or non-proteinaceous derivatizing agent, fusions to heterologous polypeptide sequences, and post-translational modifications. Covalent modified polypeptides, e.g. of a specified sequence, still have the ability bind to the human NogoA, human NiG or human NogoA_{—}342-357 by crosslinking. Covalent modifications are traditionally introduced by reacting targeted amino, acid residues with an organic derivatizing agent that is capable of reacting with selected sides or terminal residues, or by harnessing mechanisms of post-translational modifications that function in selected recombinant host cells. Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deaminated under mildly acidic conditions. Other post-translational modifications include hydroxylation of proline and lysine, phosphorylation of hydroxyl groups of seryl, tyrosine or threonyl residues, methylation of the α-amino groups of lysine, arginine, and histidine side chains, see e.g. T. E. Creighton, Proteins; Structure and Molecular Properties, W. H. Freeman & Co., San Francisco, pp. 79-86 (1983). Covalent modifications e.g. include fusion proteins comprising a polypeptide according to the present invention, e.g. of a specified sequence and their amino acid sequence variants, such as immunoadhesions, and N-terminal fusions to heterologous signal sequences.

“Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- or C-terminal extensions nor insertions shall be construed as reducing identity or homology. Methods and computer programs for the alignment are well known.

“Amino acid(s)” refer to all naturally occurring L-α-amino acids, e.g. and including D-amino acids. The amino acids are identified by either the well known single-letter or three-letter designations.

The term “amino acid sequence variant” refers to molecules with some differences, in their amino acid sequences as compared to a polypeptide according to the present invention, e.g. of a specified sequence. Amino acid sequence variants of a polypeptide according to the present invention, e.g. of a specified sequence, still have the ability to bind to human NogoA or human NiG or more preferably to NogoA_{—}342-357. Substitutional variants are those that have at least one amino acid residue removed and a different amino acid inserted in its place at the same position in a polypeptide according to the present invention, e.g. of a specified sequence. These substitutions may be single, where only one amino acid in the molecule has been substituted, or they may be multiple, where two or more amino acids have been substituted in the same molecule. Insertional variants are those with one or more amino acids inserted immediately adjacent to an amino acid at a particular position in a polypeptide according to the present invention, e.g. of a specified sequence. Immediately adjacent to an amino acid means connected to either the α-carboxy or α-amino functional group of the amino acid. Deletional variants are those with one or more amino acids in a polypeptide according to the present invention, e.g. of a specified sequence, removed. Ordinarily, deletional variants will have one or two amino acids deleted in a particular region of the molecule.

A binding molecule of the invention may be produced by recombinant DNA techniques. In view of this, one or more DNA molecules encoding the binding molecule must be constructed, placed under appropriate control sequences and transferred into a suitable host organism for expression.

In a very general manner, there are accordingly provided
(i) DNA molecules encoding a single domain Binding Molecule of the invention, a single chain Binding Molecule of the invention, a heavy or light chain or fragments thereof of a Binding Molecule of the invention; and
(ii) the use of the DNA molecules of the invention for the production of a Binding Molecule of the invention by recombinant means.

The present state of the art is such that the skilled person will be able to synthesize the DNA molecules of the invention given the information provided herein i.e. the amino acid sequences of the hypervariable regions and the DNA sequences coding for them. A method for constructing a variable domain gene is for example described in EP 239 400 and may be briefly summarized as follows: A gene encoding a variable domain of a monoclonal antibody of whatever specificity is cloned. The DNA segments encoding the framework and hypervariable regions are determined and the DNA segments encoding the hypervariable regions are removed so that the DNA segments encoding the framework regions are fused together with suitable restriction sites at the junctions. The restriction sites may be generated at the appropriate positions by mutagenesis of the DNA molecule by standard procedures. Double stranded synthetic CDR cassettes are prepared by DNA synthesis according to the sequences given CDR-H1-3A6, CDR-H2-3A6, CDR-H3-3A6, CDR-L1-3A6, CDR-L2-3A6 and CDR-L3-3A6 above. These cassettes are provided with sticky ends so that they can be ligated at the junctions to the framework by standard protocol for achieving a DNA molecule encoding an immunoglobulin variable domain.

Furthermore, it is not necessary to have access to the mRNA from a producing hybridoma cell line in order to obtain a DNA construct coding for the monoclonal antibodies of the Invention. Thus PCT application WO 90/07861 gives full instructions for the production of a monoclonal antibody by recombinant DNA techniques given only written information as to the nucleotide sequence of the gene.

The method comprises the synthesis of a number of oligonucleotides, their amplification by the PCR method, and their splicing to give the desired DNA sequence.

Expression vectors comprising a suitable promoter or genes encoding heavy and tight chain constant parts are publicly available. Thus, once a DNA molecule of the invention is prepared it may be conveniently transferred in an appropriate expression vector.

DNA molecules encoding single chain antibodies may also be prepared by standard methods, for example, as described in WO 88/1649.

In a particular embodiment of the invention, the recombinant means for the production of some of the Binding Molecules of the invention includes first and second DNA constructs as described below:

The first DNA construct encodes a heavy chain or fragment thereof and comprises
a) a first part which encodes a variable domain comprising alternatively framework and hypervariable regions, said hypervariable regions comprising in sequence DNA-CDR-H1-3A6 (SEQ ID NO: 14), DNA-CDR-H2-3A6 (SEQ ID NO: 15) and DNA-CDR-H3-3A6 (SEQ ID NO: 16); this first part starting with a codon encoding the first amino acid of the variable domain and ending with a codon encoding the last amino acid of the variable domain, and
b) a second part encoding a heavy chain constant part or fragment thereof which starts with a codon encoding the first amino acid of the constant part of the heavy chain and ends with a codon encoding the last amino acid of the constant part or fragment thereof, followed by a non-sense codon.

Preferably, the second part encodes the constant part of a human heavy chain, more preferably the constant part of the human γ4 chain. This second part may be a DNA fragment of genomic origin (comprising introns) or a cDNA fragment (without introns).

The second DNA construct encodes a light chain or fragment thereof and comprises
a) a first part which encodes a variable domain comprising alternatively framework and hypervariable regions; said hypervariable regions comprising in sequence DNA-CDR-L1-3A6 (SEQ ID NO: 17), DNA-CDR-L2-3A6 (SEQ ID NO: 18) and DNA-CDR-L3-3A6 (SEQ ID NO: 19), this first part starting with a codon encoding the first amino acid of the variable domain and ending with a codon encoding the last amino acid of the variable domain, and
b) a second part encoding a light chain constant part or fragment thereof which starts with a codon encoding the first amino acid of the constant part of the light chain and, ends with a codon encoding the last amino acid of the constant part or fragment thereof followed by a non-sense codon.

Preferably, the second part encodes the constant part of a human light chain, more preferably the constant part of the human κ chain.

The first or second DNA construct advantageously comprises a third part which is located upstream of the first part and which encodes part of a leader peptide; this third part starting with the codon encoding the first amino acid and ending with the last amino acid of the leader peptide. This peptide is required for secretion of the chains by the host organism in which they are expressed and is subsequently removed by the host organism. Preferably, the third part of the first DNA construct encodes a leader peptide having an amino acid sequence substantially identical to the amino acid sequence of the heavy chain leader sequence as shown in SEQ ID NO: 21 (starting with the amino acid at position -19 and ending with the amino acid at position -1). Also preferably, the third part of the second DNA construct encodes a leader peptide having an amino acid sequence as shown in SEQ ID NO: 23 (light chain, starting with the amino acid at position -18 and ending with the amino acid at position -1).

Each of the DNA constructs are placed under the control of suitable control sequences, in particular under the control of a suitable promoter. Any kind of promoter may be used, provided that it is adapted to the host organism in which the DNA constructs will be transferred for expression. However, if expression is to take place in a mammalian cell, it is particularly preferred to use the promoter of an immunoglobulin gene.

The desired antibody may be produced in a cell culture or in a transgenic animal. A suitable transgenic animal may be obtained according to standard methods which include micro injecting into eggs the first and second DNA constructs placed under suitable control sequences transferring the so prepared eggs into appropriate pseudo-pregnant females and selecting a descendant expressing the desired antibody.

When the antibody chains have to be produced in a cell culture, the DNA constructs must first be inserted into either a single expression vector or into two separate but compatible expression vectors, the latter possibility being preferred.

Accordingly, the invention also provides an expression vector able to replicate in a prokaryotic or eukaryotic cell line which comprises at least one of the DNA constructs above described.

Each expression vector containing a DNA construct is then transferred into a suitable host organism. When the DNA constructs are separately inserted on two expression vectors, they may be transferred separately, i.e. one type of vector per cell, or co-transferred, this latter possibility being preferred. A suitable host organism may be a bacterium, a yeast or a mammalian cell line, this latter being preferred. More preferably, the mammalian cell line is of lymphoid origin e.g. a myeloma, hybridoma or a normal immortalized B-cell, but does not express any endogeneous antibody heavy or light chain.

It is also preferred that the host organism contains a large number of copies of the vectors per cell. If the host organism is a mammalian cell line, this desirable goal may be reached by amplifying the number of copies according to standard methods. Amplification methods usually consist of selecting for increased resistance to a drug, said resistance being encoded by the expression vector.

In another aspect of the invention, there is provided a process for producing a multi-chain binding molecule of the invention, which comprises (i) culturing an organism which is transformed with the first and second DNA constructs of the invention and (ii) recovering an active binding molecule of the invention from the culture.

Alternatively, the heavy and light chains may be separately recovered and reconstituted into an active binding molecule after in vitro refolding. Reconstitution methods are well-known in the art; Examples of methods are in particular provided in EP 120 674 or in EP 125 023. Therefore a process may also comprise
(i) culturing a first organism which is transformed with a first DNA construct of the invention and recovering said heavy chain or fragment thereof from the culture and
(ii) culturing a second organism which is transformed with a second DNA construct of the invention and recovering said light chain or fragment thereof from the culture and
(iii) reconstituting in vitro an active binding molecule of the invention from the heavy chain or fragment thereof obtained in (i) and the light chain or fragment thereof obtained in (ii).

In a similar manner, there is also provided a process for producing a single chain or single domain binding molecule of the invention which comprises
(i) culturing an organism which is transformed with a DNA construct respectively encoding a single chain or single domain binding molecule of the invention and
(ii) recovering said molecule from the culture.

The binding molecules of the invention exhibit very good nerve regeneration activity as shown, for example, in the granule cell neurite outgrowth model.

1. Granule Cell Neurite Outgrowth Assay (in vitro)

Brain tissue (cortex and brain stem) is taken and for each assay protein extract freshly prepared as described previously (Spillmann et al. 1998, Identification and characterization of a bovine neurite growth inhibitor (bNI-220), J Biol Chem. 1998 Jul. 24; 273(30):19283-93). A piece of frozen tissue (e.g. 0.25 g) is homogenized in 3-4 Vol of 60 mM Chaps—20 mM Tris pH 8.0-1 mM EDTA with Protease blocker (10 μg/ml Aprotinin—5 μg/ml, Leupeptin—1 μg/ml Pepstatin—1 mM PMSF) at 4° C. Homogenate is put on a rotator at 4° C. for 30 min and centrifuged at 100,000 g 45 min 4° C. in a TLA 100.3 rotor (Beckman TL-100 ultracentrifuge). From supernatant the protein concentration is determined using BioRad. Cerebellar granule cells are purified from trypsin dissociates of postnatal day 5-7 rat cerebellar tissue as described previously (Niederost et al 1999, Bovine CNS myelin contains neurite growth-inhibitory activity associated with chondroitin sulfate proteoglycans, J Neurosci. 1999 Oct. 15; 19(20):8979-89). The binding molecules of the invention are then pre-incubated for 30 min on the test substrate and removed before the cells are added. Cerebellar granule cells are added and incubated for 24 hours. To stop the experiment, 2 ml of 4 % buffered formaldehyde is slowly added to the culture dishes. Monkey brain membrane protein extract prepared as described above was adsorbed overnight at 15 μg protein per cm² culture dish on Greiner 4-well dishes (Greiner, Nuertingen, Germany). Dishes are washed three times with warm Hank's solution before plating the neurons. Postnatal day (5-7) rat cerebellar granule cells are prepared as described above and plated at 50,000 cells/cm². Cells are cultured for 24 hr in serum-free medium, fixed, and immunostained with neurite marker MAB 1b (Chemicon monoclonal Ab, 1:200). For the staining of cell bodies DAPI (4′,6-diamidino-2-phenyl-indole, dihydrochloride, from Molecular Probes) is used after staining with MAB1b. For antibody experiments, the anti-Nogo-A mAbs or control IgG Ab are preincubated on the dishes for 30 min and subsequently removed.

Four fields at a defined distance to the edge of the well are randomly sampled for each well using a 40× objective by counting all intersections of neurites with a line placed through the center of the observation field. All cell bodies touching the line are also counted, and an index ratio of neurites per cell body is calculated for each well as reported previously (Simonen et al, 2003, Neuron 38, 201-211). All counts are done blindly on coded experiments and expressed as an index of neuritis per cell body. Results are expressed as mean index neuritis/cell body.

Enhancement of neurite outgrowth of cerebellar granule cell in the non-permissive environment of the above prepared spinal cord extract by preincubation with a binding molecule of the invention may be observed. E.g. a typical profile for the neutralizing effect of the human 3A6-IgG1 and IgG4 antibody in the granule cell neurite outgrowth model is given below:

| | | % increase |
|---|---|---|
| | Index Neurites/ | compared to |
| | cell body | control IgG |
| no antibody | 0.87 | |
| +Control IgG | 0.90 | |
| 3A6IgG1 0.1 μg/ml | 1.44 | 60% |
| 3A6 IgG4 0.1 μg/ml | 1.43 | 59% |
| +Control IgG | 0.92 | |
| 3A6IgG1 10.0 μg/ml | 1.69 | 84% |
| 3A6 IgG4 10.0 μg/ml | 1.55 | 68% |

The neutralizing activity of the molecules of the invention can also be estimated by measuring the regenerative sprouting and neurite outgrowth and functional recovery in the in vivo spinal cord injury models briefly described below.

2. Spinal Cord Injury Models in Rats and Monkeys (in vivo)

Adult Lewis rats are injured microsurgically by transecting the dorsal half of the spinal cord bilaterally at the level of the 8^{th} thoracic vertebra. Laminectomy, anesthesia and surgery are described in Schnell and Schwab 1993 (Eur. J. Neurosci. 5: 1156-1171). Neuroanatomical tracing: The motor and sensory corticospinal tract is traced by injecting the anterograde tracer biotin dextran amine (BDA) into the cortex of the side opposite to the pump or the graft. BDA is transported to the spinal cord within 10-14 days and visualized using diaminobenzidine (DAB) as a substrate as described in Brösamle et al., (2000 J. Neurosci. 20: 8061-8068).

Two weeks after a spinal cord injury destroying about 40% of the spinal cord segment T8, mainly in the dorsal half, including both main CSTs: tracing of the CST in control animals show a moderate degree of reactive sprouting of the tract. This phenomenon corresponds to the spontaneous sprouting in response to injury well known in the literature. Injured rats being treated with the binding molecules of the invention or with pumps delivering the binding molecules of the invention may show an enhanced sprouting at the lesion site and regeneration of damaged axons neurite outgrowth of damaged neurites. Moreover the animals may show improved recovery of sensorimotor functions. Such functional tests are described previously (Merkier et al, 2001, J. Neuroscience 21, 3665-73).

3. Tissue Distribution of Antibodies in Adult Monkey CNS

The antibody 3A6 is purified as IgG and concentrated to 3 mg/ml in PBS. Mouse serum derived IgG (Chemicon Int., Temecula, Calif., USA) or a mAB directed against wheat auxin (AMS Biotechnology, Oxon/UK) are used as control treatments. Two male adult macaque monkeys (Macaca fascicularis) are used in this study for intrathecal infusion.

Surgical Procedures

Anaesthesia is induced by intramuscular injection of ketamine (Ketalar®; Parke-Davis, 5 mg/kg, i.m.). Atropine is injected i.m. (0.05 mg/kg) to reduce bronchial secretions. An intravenous catheter is placed in the femoral vein for continuous perfusion with a mixture of propofol 1% (Fresenius®) and glucose 4% solution (1 volume of Propofol and 2 volumes of glucose solution), inducing a deeper anaesthesia. The animal is then placed in a stereotaxic framework. Under sterile conditions, a vertical midline skin incision is performed from C2 to Th1. The fascia cut and the spinal processes of C2 to Th1 are exposed. The paravertebral muscles are retracted and the laminae of C6, C7 and Th1 dissected. A complete C6laminectomy and an upper C7 hemilaminectomy are then performed. The dura mater is exposed and incised longitudinally above the 7^{th} and the 8^{th} cervical spinal segments, corresponding to the rostral zone of the spinal portion covered by the 6^{th} cervical lamina. A polyethylene tube (10 cm long), connected to an osmotic pump (Alzet®, 2ML1; flow: 50 μg/hr) delivering the hNogo-A antibody, is inserted below the dura and pushed a few millimeter rostrally and attached to the dura with a suture. The osmotic pump is placed and secured in a cavity made in the mass of back muscles a few centimeter lower than the laminectomy, on the left side. The tube is secured along its trajectory with sutures to muscle tissue. The muscles and the skin are sutured and the animal recovered from anaesthesia usually 15-30 minutes after interruption of the venous perfusion with propofol. The animals treated post-operatively with an antibiotic (Ampiciline 10%, 30 mg/kg, s.c.). Additional doses of Carprofen are given daily during one week.

The monkeys are sacrificed 8 days after implantation of the osmotic pump. Sedation is first induced with ketamine, as mentioned above, followed by a deep anaesthesia obtained by i.p. injection of a lethal dose of pentobarbital (90 mg/kg). The animals are perfused transcardially with 0.4 litre of 0.9% saline, followed by 4 litres of fixative (4% solution of paraformaldehyde in 0.1 M phosphate buffer, pH=7.6). Perfusion is continued with 3 solutions of sucrose of increasing concentration (10% in fixative, 20 and 30% in phosphate buffer).

Histological Procedures, Immuno-Fluorescence and -Histochemistry

Brains and spinal cords of the monkeys are carefully dissected, cryo-protected in 30% sucrose and sectioned at 40 μm in a cryostate. For detection of infused mABs an anti-human secondary antibody is used (Jackson Laboratories). For double labelling, the following antibodies can be used: the rabbit AS472 (affinity purified) for endogenous Nogo-A (Chen, 2000), rabbit antibodies against GFAP for astrocytes, and a rabbit antibody, against Cathepsin D (DAKO) for lysosomal localization. All the antisera are visualized by TRITC or FITC coupled corresponding secondary antibodies, or using the ABC-DAB. system (Vector). Sections are analysed by epifluorescence on a Zeiss Axiophot or by confocal microscopy (ZEISS LSM 410).

The spinal cords are analysed at the infusion site and 6 cm caudal to it. High levels of 3A6 are present at the infusion site. In the more caudal spinal cord, central canal and cord surface are strongly labelled, whereas grey and white matter show a more homogenous labelling, which, however, is specific and clearly over background. A similar situation is present in the forebrain with strong labelling of surface and ventricles and good penetration of the Nogo-A antibody into the parenchyma.

These experiments show that spinal intrathecal infusion of antibodies against a CNS cell surface antigen lead to a good distribution of the antibody through the CSF circulation in the inner (ventricles, central canal) and outer liquor spaces. The IgG antibodies penetrate well Into the brain and spinal cord tissue. Whereas the control IgG is washed out rapidly, the antibody against Nogo-A are retained in the tissue.

4. Tests for Nerve Repair and Functional Improvement in Spinal Lesions in Monkeys

Anaesthesia is induced by intramuscular injection of ketamine (Ketalar®; Parke-Davis, 5 mg/kg, i.m.). Atropine is injected i.m. (0.05 mg/kg) to reduce bronchial secretions. An intravenous catheter is placed in the femoral vein for continuous perfusion with a mixture of propofol 1% (Fresenius®) and glucose 4% solution (1 volume of Propofol and 2 volumes of glucose solution), inducing a deeper anaesthesia. The animal is then placed in a stereotaxic framework. Under sterile conditions, a vertical midline skin incision is performed from C2 to Th1. The fascia cut and the spinal processes of C2 to Th1 are exposed. The paravertebral muscles are retracted and the laminae of C6, C7 and Th1 dissected. A complete C6 laminectomy and an upper C7 hemilaminectomy are then performed. In order to deliver the molecules in close proximity of the lesion, the free tip of a polyethylene tube attached to the pump is fixed under the dura a few millimeter rostrally to the lesion.

Behavioural manual dexterity tests can be performed according to the published procedure. Manual dexterity is trained by placing the monkey seated in a primate chair in front of a Perspex modified “Brinkman board” (10 cm×20 cm) containing 50 holes randomly distributed; 25 holes being oriented horizontally and 25 vertically {Liu, 1999 15428/id; Rouiller, 1998 13239/id}. 2.7. The regeneration and sprouting of fibers can be assessed as described. The anterograde tracer injected in the right hemisphere is Biotinylated Dextran Amine (BDA, Molecular Probe®, 10% in saline). In the left hemisphere, the fluorescent anterograde tracer Fluorescein Dextran (Molecular Probe®, 10% in saline) is injected. Histological processing to visualise the tracers can be performed as described in details previously {Rouiller, 1994 8322/id}.

Therefore the invention also provides
(i) the use of the binding molecules of the invention in the nerve repair of a mammalian nervous system, in particular human nervous system,
(ii) a method of repairing nerves of a mammalian nervous system, in particular human nervous system which comprises administering an effective amount of the binding molecules of the invention to a patient in need of such treatment, or
(iii) a pharmaceutical composition for nerve repair of a mammalian nervous system, in particular human nervous system which comprises the binding molecules of the invention and a pharmaceutically acceptable carrier or diluent.

In particular, the binding molecules of the invention are useful for axonal regeneration and improved sprouting after nerve fiber damage. Thus the molecules of the invention have a wide utility in particular for human subjects. For example the binding molecule of the invention are useful in the treatment of various diseases of the peripheral (PNS) and central (CNS) nervous system, i.e. more particularly in neurodegenerative diseases such as Alzheimer disease, Parkinson disease, Amyotrophic lateral sclerosis (ALS), Lewy like pathologies or other dementia in general, diseases following cranial, cerebral or spinal trauma, stroke or a demyellating disease. Such demyelinating diseases include, but are not limited to, multiple sclerosis, monophasic demyelination, encephalomyelitis, multifocal leukoencephalopathy, panencephalitis, Marchiafava-Bignami disease, pontine myelmolysis, adrenoleukodystrophy, Pelizaeus-Merzbacher disease, Spongy degeneration, Alexander's disease, Canavan's disease, metachromatic leukodystrophy and Krabbe's disease, in one example, administration of the binding molecules of the invention can be used to treat a demyelinating disease associated with NogoA protein. In another example, cells which express the binding molecules of the invention may be transplanted to a site spinal cord injury to facilitate axonal growth throughout the injured site. Such transplanted cells would provide a means for restoring spinal cord function following injury or trauma. Such cells could include olfactory ensheathing cells and stem cells of different lineages of fetal nerve or tissue grafts.

The effect of long-term delayed Nogo-A blockade on functional recovery and neuro-anatomical plasticity in adult rats after stroke was the subject of an abstract published by Shih-Yen Tsai, Anay Pradham, Josh Rosales, Anis K. Mir, Martin E. Schwab, Gwendolyn L. Kartje in 2004. Purified anti-Amino Nogo-A antibody was administered by using osmotic pumps to adult rats 8 weeks after middle cerebral artery occlusion (MCAO). Recovery of function was examined using the skilled forelimb reaching test and the ladder rung walking test. The preliminary results showed that even when treating with anti-Amino Nogo-A blockade two months after stroke, recovery of function improved.

In addition, the Binding Molecules of the invention are useful for the treatment of degenerative ocular disorders which may directly or indirectly involve the degeneration of retinal or corneal cells including ischemic retinopathies in general, anterior ischemic optic neuropathy, all forms of optic neuritis, age-related macular degeneration, diabetic retinopathy, cystoid macular edema (CME), retinitis pigmentosa, Stargardt's disease, Best's vitelliform retinal degeneration, Leber's congenital amaurosis and other hereditary retinal degenerations, pathologic myopia, retinopathy of prematurity and Leber's hereditary optic neuropathy, the after effects of corneal transplantation or of refractive corneal surgery, and herpes keratitis.

Furthermore, the Binding Molecules of the invention are useful for the treatment of psychiatric conditions, particularly schizophrenia and depression.

For these indications, the appropriate dosage will, of course, vary depending upon, for example, the particular molecule of the invention to be employed, the mode of administration and the nature and severity of the condition being treated. In general, the dosage preferably will be in the range of 1 μg/kg/day to 1 mg/kg/day. The Binding Molecules of the invention are conveniently administered by pumps or injected as therapeutics at the lesioned site, e.g. they can be administered directly into the CNS intracranially or into the spine intrathecally to the lesioned site.

The Binding Molecules of the invention can be provided alone, or in combination, or in sequential combination with other agents. For example, the binding molecules of the invention can be administered in combination with anti-inflammatory agents such as but not limited to corticosteroids following stroke or spinal cord injury as a means for blocking further neuronal damage and inhibition of axonal regeneration, Neurotrophic factors such as NGF, BDNF or other drugs for neurodegenerative diseases such as Exelon™ or Levodopa. Other suitable combination partners for the treatment of stroke are Alteplase and Desmoteplase (DSPA, e.g. disclosed in WO90/09438). In one embodiment, the present invention provides a combination comprising a Binding Molecule of the invention and Desmoteplase, in particular for the treatment of stroke as well as pharmaceutical compositions comprising said combination. As used herein, two agents are said to be administered in combination when the two agents are administered simultaneously or are administered independently in a fashion such that the agents will act at the same time.

The structure of the active ingredients identified by code nos., generic or trade names may be taken from the actual edition of the standard compendium “The Merck Index” or from databases, e.g. Patents International (e.g. IMS World Publications) or other databases provide by IMS Health. The corresponding content thereof is hereby incorporated by reference. Any person skilled in the art is fully enabled to identify the active ingredients and, based on these references, likewise enabled to manufacture and test the pharmaceutical indications and properties in standard test models, both in vitro and in vivo.

Pharmaceutical compositions of the invention may be manufactured in conventional manner. E.g. a composition according to the invention comprising the molecules of the invention is preferably provided in lyophilized form. For immediate administration it is dissolved in a suitable aqueous carrier, for example sterile water for injection or sterile buffered physiological saline.

To aid in making up suitable compositions, the binding molecules of the invention and optionally a second drug enhancing the effect of the Binding Molecules of the invention, may be packaged separately within the same container, with instructions for mixing or concomitant administration. Optional second drug candidates are provided above.

The synergistic effect of a combination of the binding molecules of the invention and growth factors such as NGF may be demonstrated in vivo by the spinal cord injury models.

The invention will be more fully understood by reference to the following examples. They should not, however, be construed as limiting the scope of the invention.

In the following examples all temperatures are in degree Celsius (° C.).

The monoclonal antibody of attention in the Examples is a Binding Molecule according to the present invention comprising the variable part of the light chain (SEQ ID NO: 3) and the variable part of the heavy chain (SEQ ID NO: 2).

The following abbreviations are used:
ELISA enzyme linked Immuno-sorbant assay
FACS fluorescence activated cell sorting
FITC fluorescein isothiocyanate
FBS foetal bovine serum
HCMV human cytomegalovirus promoter
IgG Immunoglobulin isotype G
MAb monoclonal antibody
PBS phosphate-buffered saline
PCR polymerase chain reaction

### EXAMPLE 1

### Methods

Generation of human Nogo-A expression constructs (pRK7-hNogo-A): A human cDNA library constructed in lambda gt10 (Clontech) is screened with duplicate filter sets using standard procedures. Fragments of human Nogo-A are amplified by PCR from human whole brain cDNA (Clontech) using a standard protocol and subsequently cloned into pBluescript, digested and. isolated, or used as screening probes directly. A 400 bp XhoI/SmaI fragment is used as 5′ probe, the 3′ probe is amplified with primers CA-NA-2F: 5′-AAG CAC CAT TGA ATT CTG CAG TTC C-3′ (SEQ ID NO: 29) and CA-NA-3R: 5′-AAC TGC AGT ACT GAG CTC CTC CAT CTG C-3′ (SEQ ID NO: 30). Positive clones are isolated, subcloned and sequence confirmed. To obtain a full length human Nogo-A cDNA, overlapping clones are assembled using an unique EcoRI restriction site in the human Nogo-A sequence and subcloned into Bluescript vector, named Pbsnogoa. To obtain pRK7-hNogo-A, the full length cDNA was inserted into the eukaryotic expression vector pRK-7 by directional cloning.

Generation of human NiG (hNiG) expression plasmids (pET28a-hNiG) for bacterial production: A hNiG encoding DNA fragment is subcloned into BamHI/XhoI of pET28a (Novagen), after PCR amplification of the respective coding region from Pbsnogoa, in frame with the N-terminal His- and T7-tag for bacterial expression, using primer sets: forward 5′-GTC GCG GAT CCA TGG AGA CCC TTT TTG CTC TTC-3′ (SEQ ID NO: 31); reverse 5′-GTT CTC GAG TTA TGA AGT TTT ACT CAG-3′ (SEQ ID NO: 32). The final plasmid is termed pET28a-hNiG. hNiG was then expressed in E. coli BL21 pRP by induction with 1 mM isopropyl-beta-D-thiogalactopyranoside (IPGT).

Generation of mouse NiG-exon3 (mNiG-exon3) expression plasmid: The region encoding mouse exon 3 is amplified from mouse genome BAC template with primers: forward 5′-GTG CGG ATC CAT GGA TTT GAA GGA GCA GC-3′ (SEQ ID NO: 33); reverse 5′-GTT TCT CGA GTG AAG TTT TAT TCA GCT C-3′ (SEQ ID NO: 34) and subcloned into the BamHI/XhoI cloning sites of pET28a. The final plasmid construct is named pET28a-mNiG-exon3.

Cloning of monkey NiG: PolyA RNA is isolated from frozen monkey brain tissue and cDNA are synthesised using an oligo dT primer. Two overlapping fragments covering the 5′ and the 3′ region of the cDNA are amplified by PCR using sequence-specific primers and a proof-reading enzyme. The primers are designed using the known sequence of the human NiG cDNA. For amplification of the 5′ fragment the primers are 5′-TCCACCCCGGCCGCGCCCAA-3′ (SEQ ID NO: 35) and 5′-AATGATGGGGAAAGCTGTGCTG-3′ (SEQ ID NO: 36), for the 3′-fragment 5′-GGTACAAAGATTGCTTATGAAACA-3′ (SEQ ID NO: 37) and 5′-AGCAGGGCCAAGGCAATGTAGG-3′ (SEQ ID NO: 38). The two fragments are then subcloned and for each fragment at least 4 independent clones were sequenced. The full length cDNA is assembled by overlapping PCR using the primers mentioned above and the resulting product is cloned and sequenced again.

Production of recombinant NogoNiG proteins as defined above: The bacterial Nogo-A-deletion library is expressed in Escherichia coli. Proteins are extracted either by repeated sonication in sonication buffer (20 mM Tris, 50 mM NaH₂PO₄, 100 mM NaCl, pH 8.0) with 0.75 mg/ml Lysozyme, by solubilisation with B-Per™ (Pierce) or with 8 M urea. NiG expressed with pelB-leader is obtained from the periplasmic space according to the Novagen protocol for periplasmic protein purification. Supernatants of pET28-constructs are purified using the Co²⁺-Talon™ Metal Affinity Resin (Clontech) in a batch procedure. 8 M urea and B-Per™ solubilised lysates are brought to non-denaturing conditions by increasingly substituting the buffer with sonication buffer during the resin-batch procedure. Proteins are eluted with 250 mM imidazole in sonication buffer on a gravity column (BioRad). NiG proteins are further purified by gel filtration on Superdex 200 HiLoad 16/60. Supernatants of pGEX-6P constructs are purified with G-sepharose column in a batch procedure according to manufacturer indications (Amersham Pharmacia). Cleavage of GST-Nogo-66 is done by incubating solubilised GST-Nogo-66 with PreScission protease and subsequent HPLC purification. Gel electroelution is performed by preparative SDS-PAGE of 1MAC-purified recombinant Nogo and elution with BioRad Electro-Eluter into 50 mM Tris, pH 7.4, 100 mM NaCl, 0.2% (w/v) CHAPS for 1 hr at 250 mA and followed by 30 s of reversed electrode polarities. Protein concentrations of chromatography-purified proteins are determined using Pierce Coomassie Stain and BSA as standard protein. Protein concentrations of gel eluted proteins are estimated based on band intensity of silver-stained gels (Merril C R, Dunau M L, Goldman D (1981) A rapid sensitive silver stain for polypeptides in polyacrylamide gels. Analyt. Biochem. 110:201-207) with BSA as a standard.

### EXAMPLE 2

### Generation of Human 3A6-IgG mAb

Medarex Mice (Recombinantly reconstituted with human immunoglobulin genes) are immunised subcutaneously with human NiG, corresponding to a particular sequence in human Nogo-A. 3A6 monoclonal antibody was generated by standard hybridoma technology by fusion of the spleen cells of the mouse with a hybridoma cell line. Immunization of Medarex mice was carried out with human NiG 70 ug/mouse in the back of neck and flanks s.c. concentration 1.5 mg in 1.9 ml Mixed V/V with TiterMax Adjuvant. Injection of 180 μl s.c/mouse and subsequently boosted several times. Determination of anti-Nogo-A Ab titers in serum with ELISA was carried out in 96 well plates were coated with 8 ug/ml human NiG in PBS (100 μl/well) incubated 4 hours at room temperature (RT). Plates were flicked and refilled with 200 μl/well blocking buffer (PBS+5% BSA), covered and incubated 1 h at RT or overnight at 4 degrees, then washed 4 times with tapwater, refilled with PBS and flicked. Mouse serum was diluted in PBS+10% FCS (100 μl/well), and incubated 2 h at RT or overnight at 4 degrees. Dilutions of mice sera used: 1:100, 1:1000, 1:10000, 1:30000. Wash step was repeated. Goat F(ab′)2 anti-human IgG Fc specific HRP conjugate Ab was diluted in PBS/0.1% BSA/0.1% Nonidet 40 (100 μl/well) and incubated 2 h at RT or overnight at 4 degrees. Wash step was repeated. 100 μl/well BM blue POD substrate were added and incubated in the dark at room temperature 15 minutes and 50 μl/well 1M H2SO4 was added to stop HPR substrate reaction. The O:D was determinated using a microplate reader set at 450 nm. Screening of Hybridomas and clones with ELISA was carried out as described above. Human NiG (8 μg/ml, E. coli). IgG isotyping with ELISA. Experiments were carried out to determine the IgG subclass of the antibodies. Plates were coated with human NiG and culture supernatants were used at dilutions of 1:10 to 1:100. The reactivity of the antibodies was evaluated by using a panel of mouse anti-human IgG subclass (IgG1, IgG2, IgG3, IgG4) HRP conjugated mAbs by incubation for 4 h. anti-MCP1 IgG1 mAb was used as positive control. The Elisa was carried as described above. Generation of Hybridomas was done from mouse with the highest serum titers against human NiG in ELISA and was selected for fusion. Mouse was sacrificed by CO2 inhalation. Spleen taken aseptically and single cell suspension was made. Wash in PBS calcium, magnesium free. Mouse myeloma cells (PAIO) were washed in PBS. Equal numbers of mouse spleen cells 50 million were added with mouse myeloma cells and spin at RT for 10 min. 900 RPM. Supernatant withdrawn carefully and completely. Add dropwise 1 ml PEG 4000 as fusion agent (50:50 in PBS) under light agitation over 2-3 min. at RT. Shaken gently in water bath at 37 degrees for 90 seconds. Add dropwise 5 to 10 ml RPMI 1640 medium over 5 min. to dilute out the PEG, leave at RT for 10 min. Add another 20 ml serum free medium and centrifuge. Resuspend in appropriate amount of HAT medium (RPMI+10% FCS+20 ml/liter 50×HAT. Fused cells were plated out 100 μl/well, into wells containing a feeder layer of peritoneal cells from Balb/c mice (1 ml/well). Preparation of mouse peritoneal cells is carried out 24 hours earlier and 1 ml cultures in Hat medium, 24 wells Costar plate were prepared. The yield from one mouse is sufficient for one 24 well plate=approx. 2000 cells/well. Following sacrifice, the peritoneal cavity is washed out with 5 ml of 0.34M sucrose, using a 10 ml syringe and 18 gauge needle. Groups of 6 mice are collected into 1 tube, centrifuged, resuspended in HAT medium and aliqouted into wells. Culture medium was RPMI1640 with Glutamax, containing 100 μM hypoxanthine, 1.6 μM Thymidine, 0.4 μM Aminoptrin, 50 μM beta-mercaptoethanol, 50 μg/ml Gentamycin, 10% heat inactivated FCS. Medium was exchanged 50% every 3^{rd} or 4^{th} day depending on growth rate and appearance of hybridomas and after 14 days HAT medium was exchanged for HT medium by leaving out Aminoptrin. Screening of hybridomas and done generation Supernatants were tested when wells had reached approx. 80% confluency at a dilution of 1:3 in ELISA as described above. Single cell cloning was carried out from hybridomas that were positive for anti-Nogo Ab by limiting dilution. Cloning was carried out by limiting dilution, plating out 0.5 cells/100 μl/well. 4×96, 100 μl wells are set up. Mouse PE cells were used for feeder layer. Clonal growth was checked microscopically, 100 μl medium was added the day before screening is carried out. Growth rates of individual hybridomas vary, but ca. 10 days are needed for cultures to be dense enough, to yield sufficient antibody. Screening of supernatants was done at dilution of 1:10, 1:100. Culture expansion of positive clones was carried out and adapted to low serum conditions 1% for production in roller bottles and purification was done from culture supernatants in using protein A affinity.

### EXAMPLE 3

### Production and Purification of Mouse 3A6 mAb and Fab 3A6

Protein A Sepharose CI-4B column was used (Pharmacia; 11 cm bed height). Briefly, the culture supernatant after pH correction to 8.1 is loaded at 4 ml/min and the column washed to base-line at 8 ml/min using 100 mM Na₂HPO₄, pH 8.1. Bound material is finally eluted at 8 ml/min using 50 mM NaH₂PO₄, pH 3.0,140 mM NaCl and immediately neutralized (pH 7.0) with 5 N NaOH and sterile filtered. Absorbance is monitored at 280 nm. Portion of the purified material are eventually further concentrated by ultrafiltration and/or dialyzed against PBS. All the buffers used in the purification are filtered on a 10 kDa ULTRASETTE™ tangential flow device (Filtron Technology Corporation) in order to remove possible endotoxin contaminations. For the same reason the protein A resin is extensively washed with 20% ethanol and all tubings/pumps treated with 0.1 M NaOH prior to use. Protein concentration is measured spectrophotometrically at 280 nm using a reference absorption of 1.35 for 1 mg/ml. Purity is routinely assessed by SDS-PAGE under reducing conditions using 4-20% Novex gradient gels. Endotoxin content is measured by the classical Limulus Amoebocyte Lysate (LAL) reaction according to the manufacturer instructions (Endotell AG, Allschwil, Switzerland).

Generation of F_{ab} fragments: A portion of mouse 3A6 mAb is extensively dialyzed against 100 mM Na-actetate, pH 5.5, 2 mM EDTA and adjusted to a concentration of 6 mg/ml. F_{ab} fragments are generated by papain digestion (1:200 w/w ratio) in the presence of 0.25 mM cysteine. The reaction is allowed to proceed for 16 hours at 37° C. and then stopped by the addition of the specific papain inhibitor E64 (N-[N-(L-3-trans-carboxirane-2-carbonyl)-L-leucyl]-agmatine) in large excess (10 μM). The digested antibody is then passed over a column of protein A Sepharose Fast Flow in order to remove intact material and Fc fragments. The F_{ab} fraction is extensively dialysed against PBS and concentrated to about 3 mg/ml. (Papain and E64 are from Roche Molecular Biochemicals).

### EXAMPLE 4

### HPLC, Mass Spectrometry and N-Terminal Amino Acid Sequencing of V_{L} and V_{H} Region

a) Reduction and Alkylation: Purified, dried 3A6 antibody are dissolved in 40 μl of 8M urea, 0.4M NH₄HCO₃, pH 8.3. 60 μg DTT (Calbiochem), pre-dissolved in 10 μl of the same buffer as the protein, are added. Reduction is performed at 50° C. for 30 min under argon (100 fold molar excess of DTT over protein thiols). After reduction, the sample is cooled to room temperature. 304 μg of iodoacetamide (Sigma Ultra, I-1149) dissolved in the same buffer as the protein is added. Carboxamidomethylation is carried out at room temperature for 15 min in the dark. 1 μl β-mercaptoethanol is added to quench the reaction.
b) Isolation of Heavy- and Light-Chain: Carboxamidomethylated heavy and light chains of antibody are isolated by Reverse Phase High Pressure Liquid Chromatography (RP-HPLC) on a Hewlett Packard 1090M HPLC System with DR5 pumping system and diode-array UV detector. The conditions for chromatography are: PerSeptive Biosystems Poros 2.1×100 mm column packed with R1/H material; flow is 0.5 ml/min; solvents: (A) 0.1% TFA in water and (B) 0.09% TFA/acetonitril/water 9:1; gradient 25-70% B in 8 minutes at 80° C.; detection at 218/280 nm.
c) LC-ESI-MS: Mass spectrometry is carried out using a Q-T of (Micromass, Manchester, UK) quadrupole time-of-flight hybrid tandem mass spectrometer equipped with a Micromass Z-type electrospray ionization source (ESI). Acquisition mass range is typically m/z 500-2000. Data are recorded and processed using MassLynx software. Calibration of the 500-2500 m/z scale is achieved by using the multiple-charged ion peaks of horse heart myoglobin (MW 16951.5).
d) HPLC-MS of heavy and light chain: Separation of reduced and carboxamidomethylated heavy and light chain is performed on a HP1100 HPLC system (Hewlett Packard, Palo-Alto, Calif., USA) employing a 1 mm×150 mm LC Packings column packed with Perseptive Biosystems POROS R1/H. The column is held at 60° C. Sample volumes of 10 μl are injected onto the column using a CTC PAL autosampler (CTC, Zwingen, Switzerland) fitted with a Valco model C6UW HPLC valve (Valco, Houston, Tex., USA) and a 10 μl injection loop. HPLC was controlled by MassLynx software (Micromass, Manchester, UK). UV detection is at 214 nm. Eluent A is water containing 0.05% TFA. Eluent B is a 1:9 mixture of water; acetonitrile containing 0.045% TFA. A gradient from 20% B to 90% B is run in 20 minutes at 80° C. The flow rate is typically 60 μl/min. The total flow from the LC system is introduced into the UV detection cell, then the ESI source without any splitting. The HPLC system is controlled and the signal from the UV detector is processed using MassLynx software (Micromass, Manchester, UK). The following 5 signals are detected:

TABLE 1

| Measured: | Signal interpretation |
|---|---|
| A = 50614.2 Da | H-Chain with carboxamidomethyl-cysteine (CAMCys) |
| B = 5077645 Da | Signal A + 162 Da (=hexose) |
| E = 23727.8 Da | L-Chain with CAMCys |

d) N-terminal amino acid sequencing of V_{L} and V_{H} regions: Collected H+L chains peaks form HPLC are used for sequence analysis. Amino acid sequences are determined on a Hewlett Packard G1000A N-terminal Protein Sequencing System. The system performs automated Edman chemistry on protein samples retained on miniature adsorptive biphasic columns. An optimized chemistry method (double couple 3.0) is used to enhance chemical efficiency, minimize lags and herewith extend sequence analysis to about 50 residues. Analysis of PTH-amino acids is performed on an on-line Hewlett Packard HP1090 HPLC System equipped with a ternary pumping system and a narrowbore (2.1 mm×25 cm) PTH column.

Results:

From mass analysis homogeneous heavy and light chain of mouse 3A6-IgG1 are determined. The H-chain is single glycosylated. Total mass analysis of heavy and light chain shows a single mass for both chains. HPLC chromatography of mouse 3A6-IgG1 shows a single peak. After HPLC purification followed by reduction and alkylation pure heavy and light chain are available. N-terminal sequence degradation is performed on light-chain and heavy-chain. Amino acids from the N-terminal sequence of L-chain and H-chain are identified by sequence degradation.

Light Chain
EIVLTQSPATLSLSPGERATLSCRASQSVS
Heavy Chain
EVQLVESGGGLVQPGGSLRLSCAASGFTF

### EXAMPLE 5

### Cloning of the Heavy and Light Chain Genes of Human 3A6 mAb

Total RNA is prepared from 10⁷ hybridoma cells (clone 3A6) using TriPure reagent (Roche diagnostics, Germany, Cat. # 1667157) according to the manufacturers instructions. For cDNA synthesis, mRNA is isolated from above prepared total RNA using Oligotex Resin (Qiagen, Germany, cat. # 70022).

cDNA is generated by reverse transcription using the following conditions: 2 μl mRNA, 2 μl 10× reverse transcription buffer, 2 μl (dT)₂₀ primer (10 μM), 0.5 μl RNasin (Promega, 40 U/ml), 2 μl dNTPs (5 mM each), 1 μl Omniscript™ reverse transcriptase (Qiagen, Cat #205110), 10.5 μl ddH₂O, Reaction: 1 hr at 37° C. For PCR amplification of cDNA encoding for the V_{H} and V_{L} the proofreading enzyme ProofStart™ DNA polymerase is used.

PCR of light and heavy chain: Reaction mix: 2 μl cDNA, 5 μl 10× reaction buffer, 3 μl dNTPs (5 mM each), 2 μl 5′primer (10 μM) (see Table 2), 2 μl 3′primer (10 μM) (see Table 2), 1 μl ProofStart (Qiagen, Cat # 202203), 36 μl ddH₂O. PCR conditions: 95° C./5 min, (95° C./40 sec, 53° C./1 min, 72° C. 1 min)×35, 72° C./10 min. The resulting PCR products are ligated directly into pCRbluntTOPO (Invitrogen). The ligation mix is transfected into TOP 10 cells (Invitrogen) and several clones are picked. The nucleotide sequences of the variable part of the heavy chain of the 3A6 mAb (V-H. SEQ ID NO: 43) and of the light chain of the 3A6 mAb (V-L, SEQ ID NO: 44) cDNas are determined on an ABI sequencer. Altogether ten clones of mAb3A6 tight chain cDNAs from two independent experiments (RNA→cDNA→RT-PCR) were sequenced and aligned. The subsequent amino acid sequence of V-H and V-L are shown in SEQ ID NO: 2 (V-H) and SEQ ID NO: 3 (V-L). Primers used for PCR amplification of the V_{H} and V_{L} cDNAs; all primers are synthesized by MWG Biotech, Germany.

TABLE 2

| | | SEQ |
|---|---|---|
| Primer | Sequence | ID NO: |
| 5′-V_{L} | gctatggccATCGAAGCCCCAGCTCAG | 39 |
| leader | | |
| 3′-Cκ | ttaggaattcCTAACACTCTCCCCTGTTGAAG | 40 |
| 5′-V_{H} | aatgtcgaccATGGAGTTTGGGCTGAGCTGGG | 41 |
| leader | | |
| 3′-C_{H} | ttagTTATGGGCACGGTGGGCATGTGTGAG | 42 |
| hinge | | |

Cloning of the IgG4 Expression Vectors

Molecular Cloning of the V_{H} Region

The V_{H} cDNA is amplified by PCR from the recombinant pCRII-plasmid using the primers #1 and #2. The resulting PCR-fragment is cut with BstEII and subcloned into the HincII/BstEII site of HCcassAAL generating the intermediate plasmid nogohccass. By using the primer IgG4HC5′ an amino acid exchange (glutamine instead of aspartic acid) in the heavy chain leader in position -2 is achieved. The correct sequence is verified by automated sequencing and the fragment V_{H} cDNA is released by XbaI/BamHI digest. Ligation into BamHI/XbaI digested hcMCPfin resulted in the final AnogoHC3A6 expression construct.

Molecular Cloning of the V_{L} Region

The V_{L} cDNA is amplified by PCR from the recombinant pCRII-plasmid using the primers #3 and #4 thereby introducing a MluI and a HindIII site. By introducing the HindIII restriction site an amino acid exchange (R→K) in the joining region takes place. This results in changing the J5 type joining region into a J2 type. The resulting PCR-fragment is subcloned into pCRII-blunt and the sequence is verified. The correct fragment is released by MluI/HindIII digest and ligated into the expression vector LCvec-AAL160 thereby creating the final plasmid AnogoLC3A6.

| Primer | | | SEQ |
|---|---|---|---|
| # | Decsription | Sequence | ID NO: |
| 1 | IgG4HC5' | GAGGCAGAGGTGCAGCTGGTGGAG | |
| | | TCTGG | |
| 2 | IgG4HC3' | aaaTTGGTGGAGGCTGAGGAGACG | |
| 3 | IgG4LC5' | aaaaacgcgttgtGAAATTGTGTT | |
| | | GACACAGTCT | |
| 4 | IgG4LC3' | aaaaaagcttTGTCCCTTGGCCGA | |
| | | AGGTGATC | |

Characterization of the Human 3A6 mAb

### EXAMPLE 6

### Binding of 3A6 and Fab to Human Nogo-A Using ELISA

Greiner 96 well PS plates (#655161) are coated with 0.4-2 μg/ml Nogo protein fragments in PBS (100 μl/well) covered and incubated 4 hours at room temperature. Plates are flicked and refilled with 200 μl/well blocking buffer (PBS+2% BSA), covered and incubated, 1 h at RT or overnight at 4° C., then washed 3 times with water and 1 time with PBS. Different concentrations of human 3A6 IgG1 , IgG4 mAb or 3A6 Fab are diluted in PBS+2% BSA (100 μl/well), and incubated 2 h at RT or overnight at 4° C. Wash step is repeated and Goat anti-human IgG conjugated with horse radish peroxidase (HRP) at a dilution of 1:5000 (Jackson Immuno Research #109-036-098) or Donkey anti-human HRP at a dilution of 1:5000 (Jackson Immuno Research 709-035-149) for 3A6Fab in PBS/0.1% BSA/0.1% Nonidet 40 (100 μl/well) is added and incubated. 2 h at RT or overnight at 4° C. and wash step is repeated. HRP reaction is started by adding 100 μl/well BM blue POD (Roche #1484281) and incubated in the dark at RT for 15 minutes. H2SO4 50 μl/well 1M is added to stop HRP substrate reaction and the optical density is determinated using a microplate reader (Packard Spectra Count) set to 450 nm.

Results:

The human 3A6 IgG1, IgG4 mAbs and 3A6 Fab binds to human NiG at very low concentrations over the range 0.01-10 nM

### EXAMPLE 7

### Biosensor Affinity Measurements for Mouse 3A6-IgG1, 3A6-IgG4 and 3A6 Fab to Nogo-A Domains

The affinity of the mouse 3A6-IgG1 mAb, 3A6-IgG4 mAb, and of the 3A6 Fab are measured by surface plasmon resonance (SPR) using a BIAcore 2000 optical biosensor (Biacore, Uppsala, Sweden) according to the manufacturer's instructions. Recombinant human NiG is covalently immobilized on a flow cell of a CM5 sensor chip using amine-coupling chemistry. Briefly; the carboxymethlyladed dextran matrix is activated by injecting 35 μl of a solution containing 0.025M NHS and 0.1M EDC. For the immobilization on the sensor chip the recombinant human NiG is diluted in 0.0.1M citrate buffer at pH 4 and injected at a flow rate of 5 μl/min to achieve coupling levels allowing affinity measurements. The deactivation of the remaining NHS-ester group is performed by injection of 35 μl of 1M ethanolamine hydrochloride (pH 8.5). The surface of the sensor chip is regenerated by injecting 5 μl 0.1M HCl. For the measurement of the affinity the antibodies are injected at different concentrations, ranging from 0.50 nM to 100 nM at a flow rate of 200 μl/min. After each injection the sensor chip surface is regenerated with the injection of 10 μl 0.1M HCl without loss of binding activity on the surface. The kinetic constants, ka and kd and the affinity constants KA and KD are evaluated using the BIAevaluations 3.0 software supplied by the manufacturer.

Affinity measurement in BIAcore: The kinetic and the affinity binding constants of the mouse 3A6-IgG1 mAb, 3A6-IgG4 mAb, and of the 3A6 derived monovalent Fab fragment to recombinant human NogoA are measured in real time using surface plasmon resonance (SPR) technology (Biacore). For this analysis recombinant human NiG is coupled on a sensor chip surface and different concentrations of the antibodies are injected. Kinetic parameters of the binding interactions are derived from the sensorgrams by non-linear curve fitting. The affinity constants at equilibrium to human NiG for the antibodies were in the range of KDs 0.14 nM to 2.7 nM for 3A6-IgG4, 3A6-IgG1, 3A6 Fab

### EXAMPLE 8

### 3A6 mAb Epitope Identification with Pepspot Analysis

Pepspot membrane is purchased from Jerini Peptide Technologies, Berlin, Germany. Before the first incubation the membrane is rinsed with ethanol for 1 minute and three times with TBS for 10 minutes. Before each incubation with the first antibody the membrane is incubated in blocking buffer over night at 4° C. After washing for 10 minutes with TBS-T the membrane is incubated with the first antibody in blocking buffer for 3 hours at RT. Antibody concentrations are c(3A6)=0.6 nM. After three washes with TBS-T for 10 minutes the membrane is incubated for 2 hours at RT with the corresponding second HRP-labeled antibody (goat-anti human IgG Fab2 from Jackson Immuno Research) at 1:500 000 dilution in blocking buffer. After three washes with TBS-T the membrane is incubated with the chemiluminescence detection reagent (ECL Advance, Amersham Biosciences) according to the manufacturer's instructions and exposed to film.

Western Blot Analysis with Human and Monkey Nogo-A Proteins:

The Western Blot Analysis is carried out according to standard methods. 10 ng human NiG purified from E. coli is applied to each lane, SDS-PAGE is performed and transferred to nitrocellulose membrane. Blocking is at 4° C. in blocking buffer over night. After incubation of the antibody (1 nM in 0.5% blocking buffer) with the peptide (10-, 100- and 1000-fold molar excess of peptide, Human NiG peptide epitope HNQQELPTALTKLVKED, Scrambled peptide H-ETQLAKLPVDLKTQE: Jerini Peptide Technologies, Berlin, Germany) for 1 hour at RT the membrane is added to this solution and incubated 1 hour at RT on a shaker. After three washes with TBS-T for 10 minutes the membrane is incubated for 1 hour at RT with the corresponding second HRP-labeled antibody (goat-anti human IgG Fab2 from Jackson Immuno Research) at 1:100 000 dilution in blocking buffer. After three washes with TBS-T the membrane is incubated with the chemiluminescence detection reagent (ECL Advance, Amersham Biosciences) according to the manufacturer's instructions and exposed to film for 15 seconds. The Western Blot Analysis to the cynomolgus NiG is carried out according to standard methods. Aliquots of E. coli pET28-monkey NiG cell lysates expressing monkey NiG upon induction with 1PTG are applied to each lane. As a negative control cell lysates of the same cells without induction of expression is loaded. SDS-PAGE is performed and transferred to nitrocellulose membrane. Blocking is at 4° C. in blocking buffer over night. After incubation of the antibody (1 nM in 0.5% blocking buffer, Roche Applied Science) with the peptide (10-, 100- and 1000-fold molar excess of peptide, sequence: NQQELPIALTKLVKEED, Jerini Peptide Technologies) for 1 hour at RT the membrane is added to this solution and incubated one more hour at RT on a shaker. After three washes with TBS-T for 10 minutes the membrane is incubated for 1 hour at RT with an anti human HRP-labeled secondary antibody at 1:100 000 dilution in blocking buffer. After three washes with TBS-T the membrane is incubated with the chemiluminescence detection reagent (ECL Advance, Amersham Biosciences) according to the manufacturer's instructions and exposed to film for 15 seconds.

Binding of 3A6 mAb to Human and Monkey Peptide Epitopes in ELISA

Greiner 96 well PS plates are coated with 8 μg/ml scrambled peptide H-ETQLAKLPVDLKTQE, human NiG peptide epitope 3A6IgG4 H-NQQELPTALTKLVKED and peptide epitope 3A6 IgG4 monkey NiG, H-NQQELPIALTKLVKEED in PBS (100 μg/well) covered and incubated 4 hours at room temperature. Plates are flicked and refilled with 200 μl/well blocking buffer (PBS+5% BSA), covered and incubated 1 h at RT or overnight at 4 degrees centigrade, then washed 4 times with tap water, refilled with PBS and flicked. mAb 3A6IgG4 is diluted in PBS+2% BSA (100 μl/well), and incubated 2 h at RT or overnight at 4 degrees. Dilutions of mAb: 10 nM to 0.001 nM. Wash step is repeated. 2nd.Ab is diluted in PBS/0.1% BSA/0.1% Nonidet 40 (100 μl/well) and incubated 2 h at RT or overnight at 4 degrees. Wash step is repeated. 100 μl/well BM blue POD substrate are added and incubated in the dark at room temperature 15 minutes and 50 μl/well 1M H2SO4 is added to stop HRP substrate reaction. The OD is determined using a microplate reader set to 450 nm.

Results:

Epitope mapping of the human 3A6 mAb: The epitope mapping results in a sequence ELPTALTKLV in human NiG protein.

Competition of mAb 3A6 Binding to Human NiG in Western Blot with Synthetic Peptide:

To confirm the result obtained by the pepspot technique western blot competition experiment is performed. A synthetic 16-mer containing the putative epitope sequence (NQQELPTALTKLVKED) is used to compete with full length human NiG for binding to the 3A6 antibody. Prior to incubation with membrane bound human NiG the 3A6 antibody (1 nM) is incubated for 1 hour with the synthetic peptide using different molar ratios of peptide to antibody. A 10-fold molar excess of peptide shows a significant decrease in the detected signal for human NiG (produced in E. coli). A 100-fold excess results in a further decrease of the signal, and a 1000-fold molar excess of the peptide nearly completely inhibits the binding of the 3A6 to human NiG. In contrast a 1000-fold excess of a peptide with the same amino acid content but with a different sequence (scrambled) does not have any effect on the binding of the antibody to human NiG.

mAb 3A6 Binding to Monkey NiG: Competition with Synthetic Peptide Epitope

A synthetic 17-mer containing the epitope sequence (NQQELPIALTKLVKEED) is used to compete with full length cynomolgus monkey NiG expressed in E. coli for binding to the 3A6 antibody. Prior to incubation with membrane bound monkey NiG the 3A6 antibody (1 nM) is incubated for 1 hour with the synthetic peptide using different molar ratios of peptide to antibody. A 100-fold excess results in a decrease of the signal, and a 1000-fold molar excess of the peptide substantially inhibits the binding of the 3A6 to monkey NiG. In contrast a 1000-fold excess of a peptide with the same amino acid content but with a different sequence (scrambled) does not have any effect on the binding of the antibody to human NiG.

Binding of 3A6 IgG4 to the Human and Monkey NiG Peptide Epitope in ELISA

Detailed binding analyses of the mAb to the epitope and scrambled sequence are performed using ELISA. The results show dearly that the mAb binds in a concentration dependent manner at very low concentrations (0.001 to 1.0 nM) to monkey and human peptide epitopes comparable to its KD in BIAcore for human NiG of 0.14 nM. Moreover binding is specific with no binding to the scrambled control peptide.

1-19. (canceled)

20. A human or chimeric or humanized monoclonal antibody, or a F(ab′)₂ or Fab fragment thereof, which binds the human NogoA epitope defined by amino acid sequence ELPTALTKLV.

21. An antibody or fragment thereof according to claim 20, which binds the human NogoA_{—}342-357 epitope or the epitope defined by amino acid sequence NQQELPTALTKLVKED.

22. The antibody or fragment thereof of claim 20, which binds said human NogoA epitope with a dissociation constant (Kd)<100 nM.

23. The antibody or fragment thereof of claim 21, which binds said human NogoA epitope with a dissociation constant (Kd)<100 nM.

24. The antibody or fragment thereof of claim 22, which binds said human NogoA epitope with a dissociation constant (Kd)<10 nM.

25. The antibody or fragment thereof of claim 23, which binds said human NogoA epitope with a dissociation constant (Kd)<10 nM.

26. The antibody or fragment thereof according to claim 20, which is a single chain or single domain antibody.

27. The antibody or fragment thereof according to claim 20, in which the constant part or fragment thereof of the human heavy chain is of the γ4-type and the constant part or fragment thereof of the human light chain is of the κ-type.

28. A composition comprising the antibody or fragment thereof according to claim 20.

29. A method of inducing or enhancing nerve repair comprising administering to a subject in need of nerve repair an effective amount of the antibody or fragment thereof according to claim 20.

30. A method of treatment of a disease of the peripheral or central nervous system comprising administering to a subject in need of such treatment an effective amount of the antibody or fragment thereof according to claim 20.

31. A pharmaceutical composition comprising an antibody or fragment thereof according to claim 20, in association with at least one pharmaceutically acceptable carrier or diluent.

32. An epitope of the human NogoA protein consisting of the amino acid sequence of human NogoA ELPTALTKLV.

33. An epitope of the human NogoA protein consisting of the amino acid sequence of human NogoA_{—}342-357 or consisting of amino acid sequence NQQELPTALTKLVKED.

34. A composition for inducing or enhancing nerve repair, said composition comprising the antibody or fragment thereof according to claim 20.

35. A composition for use in the treatment of disorders of the peripheral or central nervous system, said composition comprising the antibody or fragment thereof according to claim 20.
